(19) United States
(12) Patent Application Publication
(10) Pub. No.: US 2025/0222099 A1
(43) Pub. Date: Jul. 10, 2025

(54) DENDRITIC CELL TARGETING FILAMENTOUS PHAGE-BASED CANCER TREATMENT VACCINE

(71) Applicant: The University of Hong Kong, Hong Kong (HK)

(72) Inventors: Jian-Dong HUANG, Hong Kong (HK); Yee Man AU-YEUNG, Hong Kong (HK); Zheng ZENG, Hong Kong (HK)

(21) Appl. No.: 19/015,516

(22) Filed: Jan. 9, 2025

Related U.S. Application Data
(60) Provisional application No. 63/619,280, filed on Jan. 9, 2024.

(51) Int. Cl. A61K 39/385; A61K 39/00; A61P 35/00; C12N 7/00

(52) U.S. Cl. CPC A61K 39/385; A61K 39/0011; A61P 35/00; C12N 7/00; A61K 2039/545; A61K 2039/55522; A61K 2039/55544; A61K 2039/55561; A61K 2039/55566; A61K 2039/55572; A61K 2039/6031; C12N 2795/14122; C12N 2795/14134

(57) ABSTRACT

A dendritic cell (DC)-targeting M13 phage vaccine platform is provided herewith, where spy-tagged neoantigens can be attached to the phage surface with spy-catchers. By expressing DC-targeting peptides and spy-catchers as foreign antigens on the phage surface, its therapeutic and antigenic properties can be enhanced to combat solid cancers. SLS-spy catcher phage (SCP), with or without neoantigen conjugation, significantly repressed tumour growth by boosting systemic anti-tumour immunity and increasing intratumoral infiltration of both innate and adaptive immune cells. Intratumoral administration of SCP also hugely reduced PDL1 expression on tumour cells, making them more susceptible to immune attacks. Additionally, SCP administration restricted the size of blood vessels inside the tumour mass, suggesting multiple factors contribute to restrict tumour growth.

### CROSS-REFERENCE TO RELEVANT APPLICATIONS

The present application claims priority from a U.S. provisional patent application Ser. No. 63/619,280 filed Jan. 9, 2024, and the disclosures of which are incorporated by reference in their entireties.

### REFERENCE TO SEQUENCE DISCLOSURE

The sequence listing file under the file name “P3060US01_SEQ_LISTING.xml” submitted in ST.26 XML file format with a file size of 3 KB created on Jan. 9, 2025 and filed on Jan. 9, 2025 is incorporated herein by reference.

### FIELD OF THE INVENTION

The present invention generally relates to the field of phage-based cancer immunotherapy. More specifically, the present invention relates to a dendritic cell-targeting M13 phage vaccine platform, where spy-tagged neoantigens can be attached to the phage surface with spy-catchers and can be utilized as a universal cancer immunotherapy platform.

### BACKGROUND OF THE INVENTION

With the aging population, cancer presents as one of the major health concerns worldwide. Therapeutic cancer vaccines have recently been developed as a form of immunotherapy to train host immunity to attack cancer cells. With next-generation sequencing, immunogenic epitopes from cancer-specific somatic mutations, namely neoantigens, have been identified as a potential target for cancer vaccines. Yet, the production of these vaccines could take approximately 4 months, and often requires combination with adjuvants and immune checkpoint blockade therapies for optimal anti-cancer effects. Peptide and protein-based vaccines also require harsh storage conditions, increasing the production cost.

Bacteria- and virus-based vaccines have therefore come into spotlight as potential vectors for therapeutic cancer vaccines. They are easy to modify and can elicit strong immunity as mammalian pathogens, yet, safety remains a major concern. On the other hand, while bacteriophages have long been considered as an anti-bacterial agent, increasing attention has been drawn to use phage-based vectors as delivery vehicles for cancer therapy due to their safety profile.

Phages are viruses that exclusively infect bacteria. Multiple studies have demonstrated their safety and potential as a clinical therapeutic agent. Bacteriophages are approved to be administered in meat and poultry products as food additives by the FDA in 2006. Meanwhile, a multiple myeloma clinical trial in 2014 demonstrated that phage idiotype vaccines are well tolerated by all study participants. Nonetheless, as a foreign pathogen, bacteriophages carry bioactive components like CpG and can trigger robust immune responses through mechanisms like Toll-like receptor pathways. Bacteriophages hence stand out as a favourable vector for cancer vaccines with their immunogenicity and high safety profile.

A large variety of studies have been conducted to engineer bacteriophages to elicit cancer-specific immunity. With phage display technology, genetic and chemical engineering, both the interior and surface of phages can be armed with therapeutic agents to serve as vectors for chemotherapy, gene therapy, anti-angiogenic, photothermal and immunotherapy. Among which, several groups have attempted to develop neoantigen-displaying phages as cancer vaccines. However, the effectiveness has been inconsistent. On the other hand, anti-tumour effects of phages may also be the size and variety limitations on proteins expressed with phage display technology.

Therefore, the present invention addresses the need of a phage vaccine with consistent effectiveness of neoantigen display and hence enhanced anti-cancer immunity, while maintaining a high cost-effectiveness by enabling mass production of the vaccine. The present invention addresses this need.

### SUMMARY OF THE INVENTION

In response to the need of a highly cost-effective, high scalability and versatile design of phage vaccine with high specificity, the present invention discloses a dendritic cell-targeting anti-cancer phage immunotherapy composition.

In a first aspect of the present invention, the dendritic cell-targeting anti-cancer phage immunotherapy composition comprises genetically engineered M13 filamentous phages and optionally neoantigens. The genetically engineered phages are designed to specifically express dendritic cell-targeting peptides (“SLS”) and synthetic spy catcher proteins; while the neoantigens are tagged with synthetic spy tag peptides which forms a fusion protein when presented to the synthetic spy catcher proteins.

In an embodiment of the first aspect of the present invention, the surface of the phage is further linked with an adjuvant.

In a further embodiment, the adjuvant is selected from tetanus endotoxin, diphtheria toxoid, monophosphoryl lipid A, CpG oligonucleotides, polyinosinic acid-polycytidylic acid (polyIC), saponin-based adjuvants, granulocyte-macrophage colony-stimulating factor (GM-CSF), or combinations thereof.

In yet another embodiment, the dendritic cell-targeting peptide comprises an amino acid sequence of SEQ ID NO. 1.

In a further embodiment, the genetically engineering phage expresses the dendritic cell-targeting peptide on its p3 site, and expresses the synthetic spy catcher protein on its p8 site.

In other embodiment, a linker is provided between the neoantigen and the synthetic spy tag peptide, and the linker is expressed to enhance antigen presentation.

In a further embodiment, the linker comprises an amino acid sequence of SEQ ID NO. 2.

A method of treating cancer or cancerous tumour in a subject is also provided herewith, comprising administering no less than two doses of the dendritic cell-targeting anti-cancer phage immunotherapy composition mentioned above. Specifically, the administering comprises no less than one subcutaneous administration and one intratumoral administration.

In an embodiment, the tumour size is reduced by 50% within 30 days from the final administration of the dendritic cell-targeting anti-cancer phage immunotherapy composition.

In another embodiment, the tumour size reduction effect sustains for at least 60 days since the final administration.

In yet another embodiment, the subject after total tumour regression shows no recurrence of tumour.

### BRIEF DESCRIPTIONS OF DRAWINGS

FIG. 1A is a schematic illustration of the design of SLS-SCP vaccine. There are three major functional elements on SCP: (1) spy catcher proteins expression on p8 positions; (2) DC-targeting peptides SLS; (3) Spy-tagged neoantigens or any proteins of interest to be connected to the phage surface by binding to spy catchers. LLSVGG cathepsin-sensitive linker is expressed between spy-tag and neoantigen sequence to enhance antigen presentation and immune activation. FIG. 1B illustrates aggregation of SCP phages after mixing with spy-tagged GFP proteins. FIG. 1C shows the flow cytometry results showing the percentage of DC2.4 cells with GFP signals after different incubation period. DC2.4 cells incubated with GFP-labelled SCP vector showed a significant increase in fluorescence signal compared to the cells incubated with GFP-labelled phages that only express spy-catchers. FIG. 1D is a comparison of the percentages of DC2.4 cells with GFP signals, after incubation with GFP-labelled SCP vector and spy catcher-expressing phages respectively under different incubation times. FIG. 1E shows confocal images of DC2.4 cells after incubation with GFP-labelled SCP vectors and spy catcher-expressing phages.

FIG. 2A illustrates the timeline for MB49 treatment model. FIG. 2B shows the tumour growth curve of mice treated by different vaccines within a 27-day window. FIG. 2C shows images of the tumours of the mice treated by two doses of intratumoral administration of SCP, which are on average 14.4 times smaller at endpoint than those that are only treated subcutaneously (p=0.0147). These tumours are 29.2 times smaller than those in mice treated by neoantigen vaccine (p=0.0005). FIG. 2C shows pictures of tumours on day 27. FIG. 2D shows the CD68 IHC staining. FIG. 2E shows the corresponding quantification of CD68 positive cells in IHC staining. FIG. 2F shows CD68 clusters which are seen in mice treated by intratumoral doses of SCP. FIG. 2G shows the results of CD31 IHC staining, showing that blood vessels in intratumorally treated group are of lesser sizes than those in PBS group.

FIG. 3A shows the timeline and vaccine designs of MB49 treatment model. FIG. 3B shows the tumour growth curve of mice treated by different vaccines. FIG. 3C shows the picture of tumours on day 27, together with comparison of tumour size for the groups with better anti-cancer effects. FIG. 3D illustrates the ELISA results on IgG humoral response against MB49 cells. FIG. 3E demonstrates IFNγ-based ELISPOT results showing the systemic cell-mediated immunity against MB49 cell lysate. FIG. 3F shows the measurement of percentage of T cells in tumour mass. FIG. 3G illustrates the phenotypic analysis of intratumoral T cells with flow cytometry.

FIG. 4A shows the timeline for timepoint experiment of MB49 treatment model. FIG. 4B illustrates the evaluation of CD45+ population in tumour mass. FIG. 4C illustrates the evaluation of CD45+CD3+ T cell population in tumour mass. FIG. 4D illustrates the evaluation of CD8+ and CD4+ T cell population in tumour mass. FIG. 4E illustrates the evaluation of CD38+ cell population in tumours. FIG. 4F illustrates the evaluation of CD3-CD11c+ DC population in tumours. FIG. 4G illustrates the evaluation of CD16+CD62L+ neutrophil population in tumours. FIG. 4H illustrates the Evaluation of PDL1 expression on tumour cells. FIG. 4I shows the humoral response against MB49 cell lysate and MB49 cells.

FIG. 5A shows the anti-tumour response of SCP vaccine on MB49 tumours seeded in Mumt and Rag1 models. FIG. 5B demonstrates MB49 tumour volumes on day 27 in different animal models with pictures of the tumours at the endpoint.

FIG. 6A is a timeline of B16F10 treatment model. FIG. 6B shows tumour growth of mice treated by different vaccines.

FIG. 7 shows a visualization of the raw data of flow cytometry results on GFP signals detected in DC2.4 cells. SCP, with or without neoantigens, repress tumour growth by eliciting multi-factorial modifications to tumour microenvironment.

FIG. 8 shows the measurements of tumour size on day 27.

FIG. 9 are pictures showing mice in SCP group on day 27. Arrow shows the mouse with tumour.

FIGS. 10A to 10C show the tumour growth for the timepoint experiments and pictures of tumours. FIGS. 10A, 10B and 10C respectively corresponds to days 16, 19 and 22.

FIG. 10D illustrates the evaluation of CD38+CD4+ or CD8+ T cell population in tumour mass.

FIG. 10E shows evaluation of CD80, CD83 and CD86 expression profiles among intratumoral DC population. FIG. 10F shows the humoral response against MB49 neoantigens administered.

FIG. 11A provides a schematic illustration of the induction of phage vectors into dendritic cells (DCs). FIG. 11B shows the expression levels of cos-stimulatory markers CD40,80 and 86 among DCs after induction.

FIG. 12A is a schematic illustration of the incubation of naïve T cells with phage-induced DCs together with the respective tumour antigens. FIG. 12B shows the tendency of the T cells co-cultured with SPC-induced DCs skewing towards of cytotoxic phenotypes, and the higher level of cytotoxic effector protein expressions. FIG. 12C tabulates the expression of pro-inflammatory genes and key immune modulators by the DCs co-cultured with SCP vectors. FIG. 12D shows the upregulation of different signalling pathways through the SCP co-culture.

FIG. 13 shows a B16F10 melanoma treatment model with more aggressive tumour growth of upon reaching a tumour size of 400 mm3, and the tumour treatment results.

FIG. 14 shows a YTN16 gastric cancer treatment model, and the tumour treatment results after a prolonged period of more than 2 months following the final intratumoral dose.

FIG. 15A shows MB49 recurrence models, illustrating (i) the initial tumour treatment results; and (ii) the protective effects of the intratumoral doses after a prolonged period of more than 2 months against the re-inoculation of MB49 cells into the same models. FIG. 15B provides the change in tumour size in all 5 MB49 recurrence models, showing little to no tumour recurrence across all mice.

FIG. 16A shows the timeline for timepoint experiment of MB49 treatment model. FIG. 16B illustrates the percentage of intratumoral CD45+ cells after the first dose of intratumoral treatment. FIG. 16C shows the tumour size over the entire course of treatment. FIG. 16D shows the population of intratumoral CD45+CD86+ cells after the first intratumoral dose.

FIG. 16E shows the CD86 staining of the tumour tissue. FIG. 16F further shows the tumour tissue under CD68 staining. FIG. 16G shows the tumour tissue under CD163 staining.

FIG. 17A shows the shift of neutrophil percentage in the intratumoral immune cells (left), and the phenotypic shift of the neutrophil under the corresponding time points (right).

FIG. 17B shows the shift of CD3+ T-cell percentage in the intratumoral immune cells. FIG. 17C illustrates the corresponding shifts in phenotypes and population of different T-cell types throughout the course after the first intratumoral dose.

FIG. 18A shows the shift of CD45+CD3-CD11c+MHC2+ cell percentage in the intratumoral immune cells (above); and the shift of CD86+ cell percentage in the DCs (below). FIG. 18B illustrates the isolation of intratutumoral DCs on day 19, co-culturing with naïve T-cells (left), the expression of MB49-targeting cytotoxic activities (middle) and the corresponding HMGB1 staining (right). FIG. 18C shows the model setup and results of OVA-expressing B16 melanoma model, using OVA proteins as a measurable antigen.

FIG. 19 tabulates the change in RNA levels of multiple pro-inflammatory functional proteins and hallmarks of immunogenic cell death with the treatment.

FIG. 20 illustrates the humoral response and systemic antigen-specific cell-mediated immune response against MB49 cells respectively, and the body weight change of the mice subject to the treatment.

FIG. 21 shows the suppression of PDL1 expression by targeting DCs as SCP leads to lower expression level compared to spy-catcher phages.

### DETAILED DESCRIPTION

As used in the disclosure herein, “SCP” refers to SLS-spy catcher phage vaccine, the phage-based vaccine composition of the present invention comprising a phage expressing spy catcher proteins allowing spy-tagged neoantigens attachment, and SLS peptide.

As used in the disclosure herein, “SLS” refers to a dendritic cell-targeting synthetic peptide, engineered to be expressible by the phage vector in the present invention to allow the neoantigen-carrying phage vector to be dendritic cell-specific targeted drug delivery.

As used in the disclosure herein, “spy catcher” refers to a protein engineered to be expressible by the phage vector. The spy catcher protein has a receptor site allowing its capturing of spy-tagged neoantigens through irreversible covalent conjugation, and hence ensuring the phage vector to carry the neoantigens to the target site.

As used in the disclosure herein, “spy” or “spy tag” refers to a short specific peptide sequence, the DNA sequence encoding which is recombinantly introduced into the sequence encoding the neoantigen of interest, such that the neoantigen is tagged by the said specific peptide sequence for bioconjugation with the spy catcher expressed on the surface of the phage vector.

As used in the disclosure herein, “DC” is the abbreviated form of “dendritic cells”.

Dendritic cells play a key role in regulation of the immune system of the human body, crucial in its initiation and modulation functions regarding immune responses. As antigen-presenting cells, in the occurrence of neoplastic tumours, DCs are able to capture the tumour antigens and present the relevant information to T cells in lymphoid tissues for the subsequent activation of cytotoxic T-lymphocytes for specific targeting and killing of tumour cells.

However, cancer tumours in general possess a wide variety of immunosuppression functions. Some examples include (i) their expression of certain DC maturation- and activation-suppression factors such as IL-10 and VEGF, promoting differentiation of immature or tolerogenic DCs; (ii) secretion of factors such as PGE2 and IDO, blocking the maturation of DCs and hence limiting its capacity to present tumor antigens and preventing full activation of cytotoxic T cells; (iii) downregulation of co-stimulatory molecules like CD40, CD80 and CD86, which are essential for T cell activation; and (iv) releasing factors that induce DC apoptosis.

As such, cancer tumours are able to evade host immune surveillance and create an advantageous environment allowing its grow and metastasis.

Therefore, by combining DC-targeting medication and phage delivery, not only is the phage vector non-pathogenic, but it can also be customized to precisely target DCs. As the phages are readily phagocytosed by the DCs, this also facilitates the presentation of neoantigens tagged by the phage vectors to the DCs for processing and downstream immunity triggering. Moreover, the structural components of the phage, for example its capsid protein, may be further tagged with adjuvants for more broad-spectrum anti-cancer therapeutic effects.

In this invention, a DC-targeting cancer vaccine platform is developed with M13 filamentous phages. The engineered phages express DC-targeting peptides (SLS) on p3 sites and spy-catcher proteins on p8 positions, allowing multiple neoantigens and proteins of interest to be linked to the phage vector. The platform is named SCP (SLS-spy catcher phage). In vitro experiments demonstrated that spy-tagged proteins can easily attach to phage vectors through simple mixing. Enhanced antigen presentation to DCs also elicited stronger anti-cancer responses. In MB49 and B16 mouse models, SCP effectively suppressed tumour growth by inducing systemic anti-tumour humoral and cell-mediated immunity. When administered intratumorally, SCP triggered particularly robust local anti-tumour response by boosting local immunity, reducing PDL1 expression, and restricting vascularization in the tumour microenvironment.

The SCP vaccine can induce robust anti-tumour immunity. It can repress tumor growth by boosting systemic anti-tumor immunity and increasing intratumoral infiltration of both innate and adaptive immune cells. The anti-tumour effect can also return back to baseline level after the tumour resolved. The vaccine action is mainly local at the tumour site, leading to minimal systemic side effects. On the other hand, SCP is produced with the phage display technology. By engineering naturally existing pathogens, SCP vaccines can be mass-produced cost-effectively.

SCP is an improvement to the current phage-based cancer vaccine produced by phage display technology. Multiple trials are conducted on using phages to generate cancer treatment vaccine, by expressing neoantigens on the surface or by expressing cancer targeting peptides. However, phage display technology has size and variation limitations, whereby only limited types of small peptides can be well displayed on the surface. Hence, by expressing spy catchers on the phage, this limitation can be overcome, and any spy-tagged proteins or peptides can be linked to the phage carrier. Then, by expressing a dendritic-cell targeting peptide on the other site of the phages, the immunogenicity and anti-cancer effectiveness of the phage vaccine can be enhanced, making it more efficient than the previous designs. Moreover, the anti-tumour effect of the phages can be amplified by linking toxins and adjuvants to the surface, for example diphtheria toxoid, to elicit robust broad-spectrum anti-cancer effects.

### EXAMPLES

### Example 1: Construction and Verification of SCP

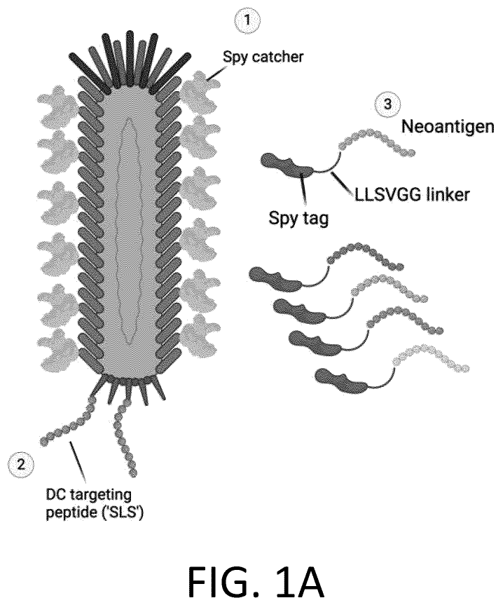

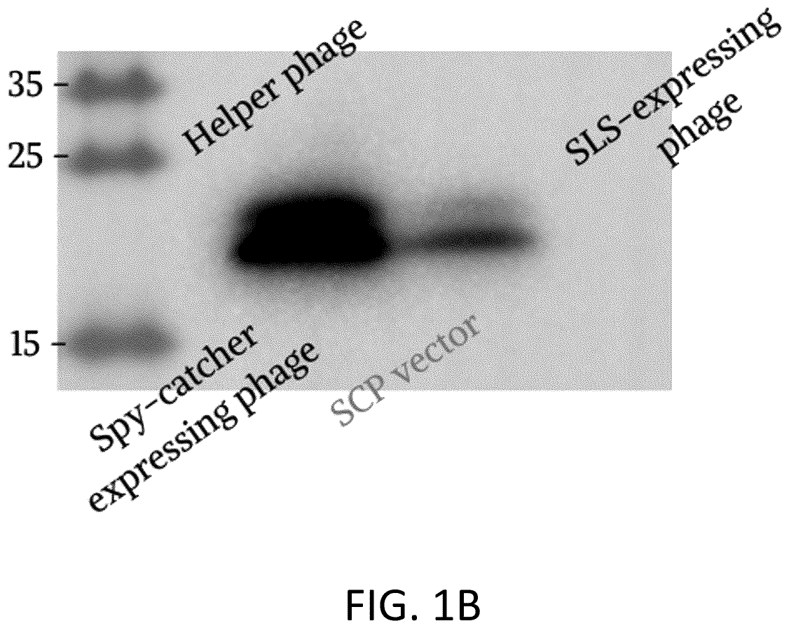

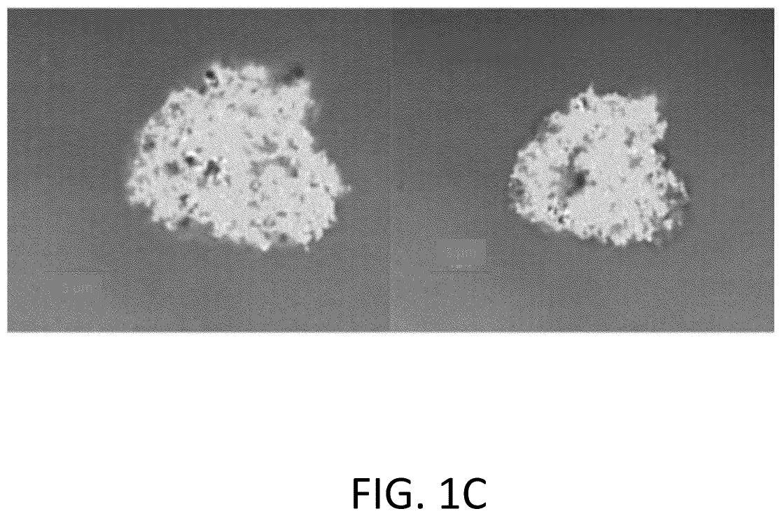

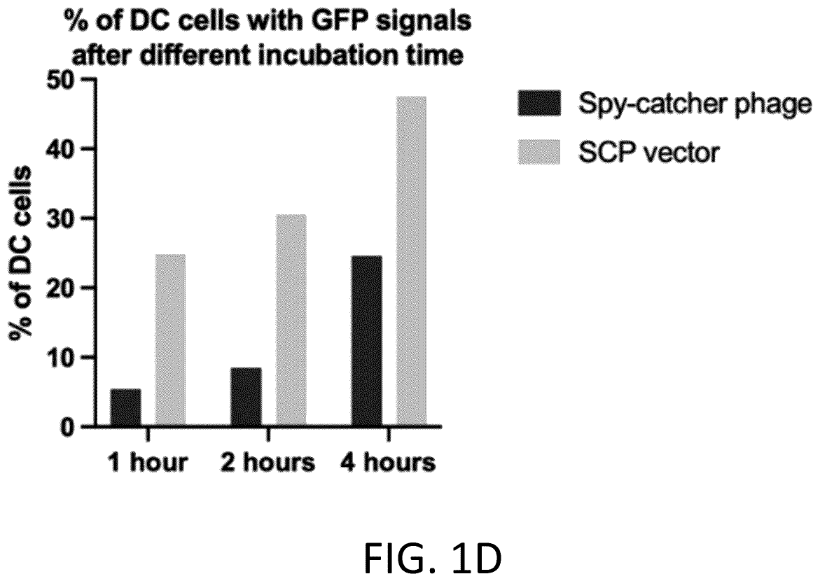

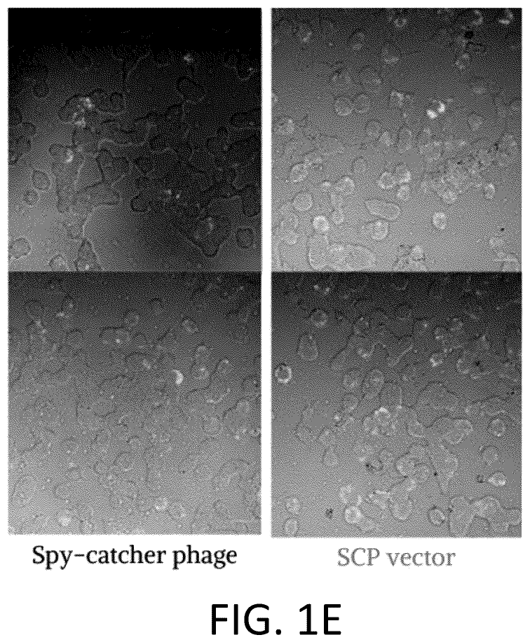

The SCP vector is first constructed based on M13 filamentous phages with phage display techniques. To enhance the antigenic characteristics of phages, SCP is designed to target dendritic cells. Three 12-mers are previously isolated from phage display library. These peptides can enhance DC antigen capturing and presentation, which thereby increase antigen immunogenicity. The sequence of one of these peptides, SLSLLTMPGNAS (SLS) (SEQ ID. NO. 1), is inserted into the pComb3 plasmid. Meanwhile, the sequence of spy catcher is inserted into pComb8 plasmid, such that spy-tagged proteins can adhere to the phage surface. The two recombinant plasmids are then transformed into XLIB E. Coli bacteria. By infecting the plasmid-carrying XLIB with M13 superphage, phages can co-express spy-catcher on p8 sites and SLS on p3 sites (FIG. 1a). On the other hand, cathepsin-sensitive linker LLSVGG (SEQ ID NO. 2) is previously screened to serve as an optimal linker that enhances cross-presentation in dendritic cells and activates both CD4 and CD8 T cells. Hence, to allow more effective release of neoantigens, the LLSVGG linker is added between the spy tag and the neoantigen.

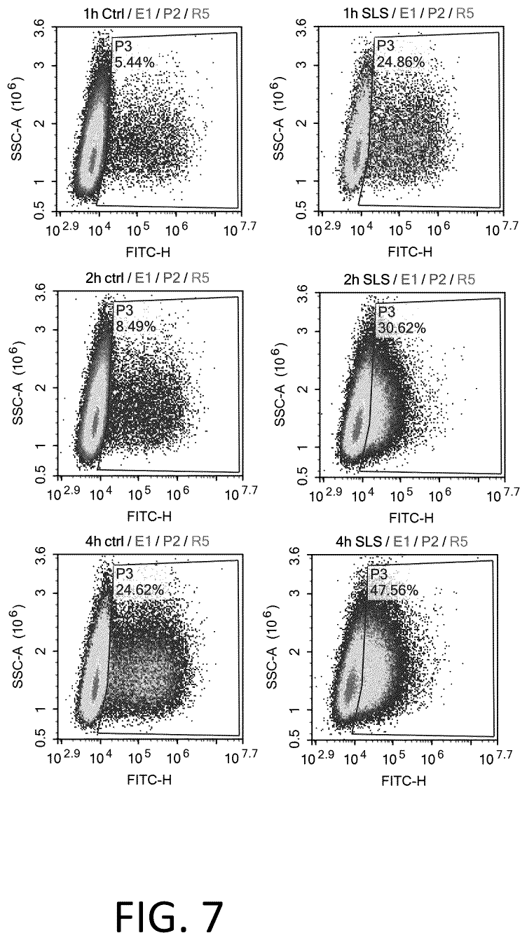

Expression of spy-catcher is first confirmed by western blot (FIG. 1B). SLS only has the size of around 1.2 kDa. Its expression and function are therefore verified with the help of GFP fluorescence. Spy-tagged GFP is first added to the SCP solution. The mixture is then concentrated with PEG-6000 and washed with PBS twice. FIG. 1B shows that spy-tagged proteins can be loaded on the phage surface just by simple mixing. The GFP-labelled phages are then incubated with DC2.4 dendritic cell line for different duration. It is observed that the fluorescence signal in SCP group is around 4.6, 3.6 and 1.9 times higher than that in the spy-catcher group at the 1, 2 and 4-hour timepoints (FIGS. 1C, 1D and 7). Hence, with the expression of SLS, a significantly higher percentage of DCs engulfed the phage vectors. The timewise changes also indicated that SLS speeded up the interaction between phages and DCs.

Z-stack on confocal imaging showed that fluorescence signals light up at different layers of the cells. This demonstrated that the phages did not just interact with DCs on the surface, but are actually engulfed by the cells. It is hence demonstrated that the two components expressed on the M13 phages are functional to amplify the antigenic effect of bacteriophages.

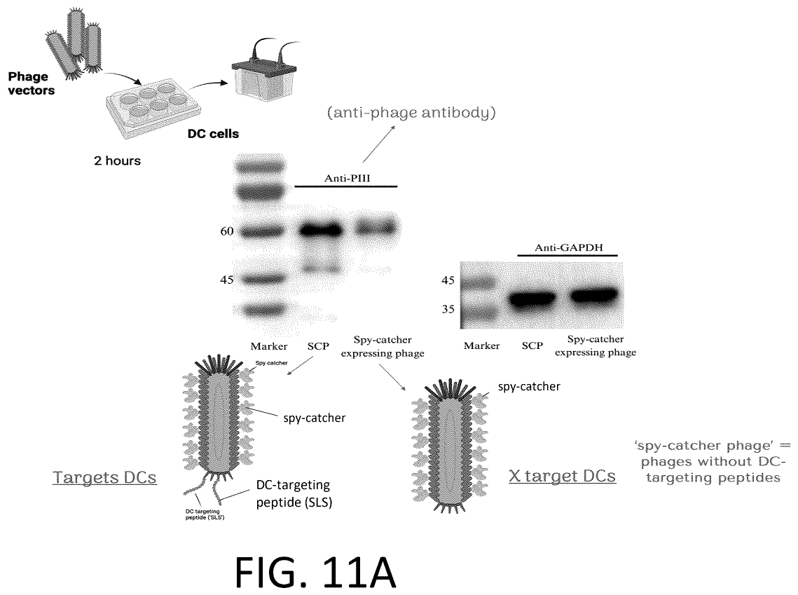

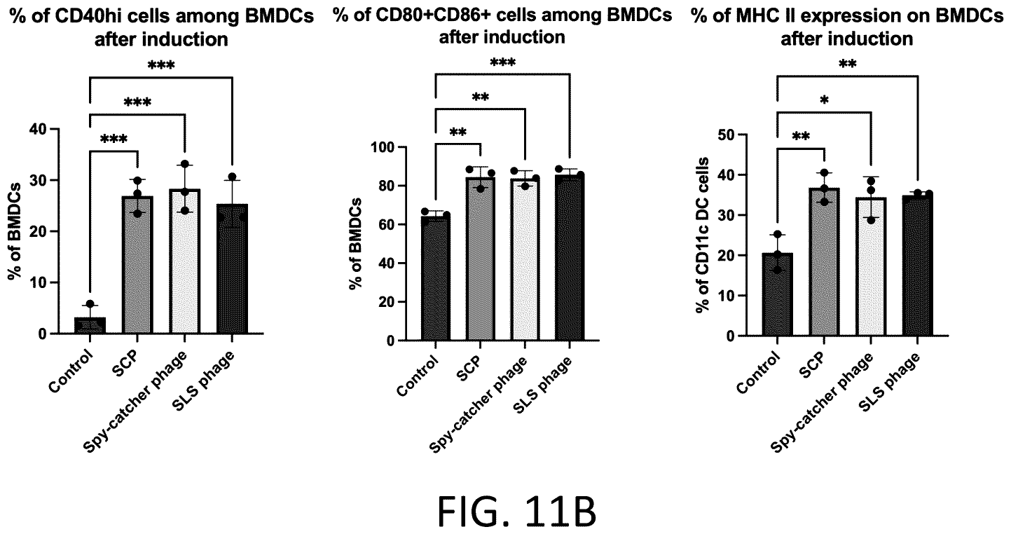

Importantly, the direct delivery of phage vectors into dendritic cells can enhance the expression of co-stimulatory markers like CD40, 80 and 86. MHC-II expression on DCs can also be upregulated, suggesting SCP vectors has the potential capability to revive the DCs in the tumour microenvironment (FIGS. 11A and 11B).

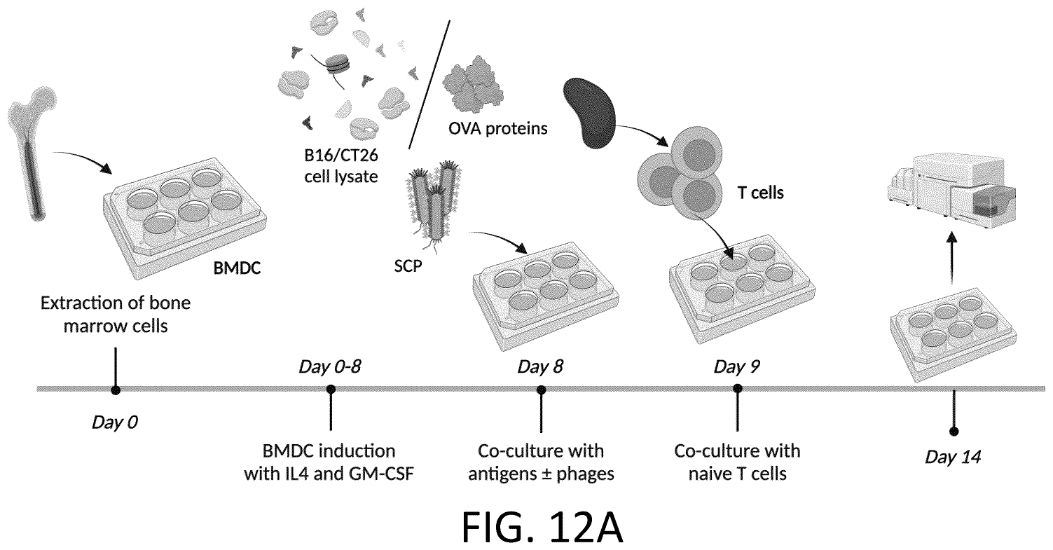

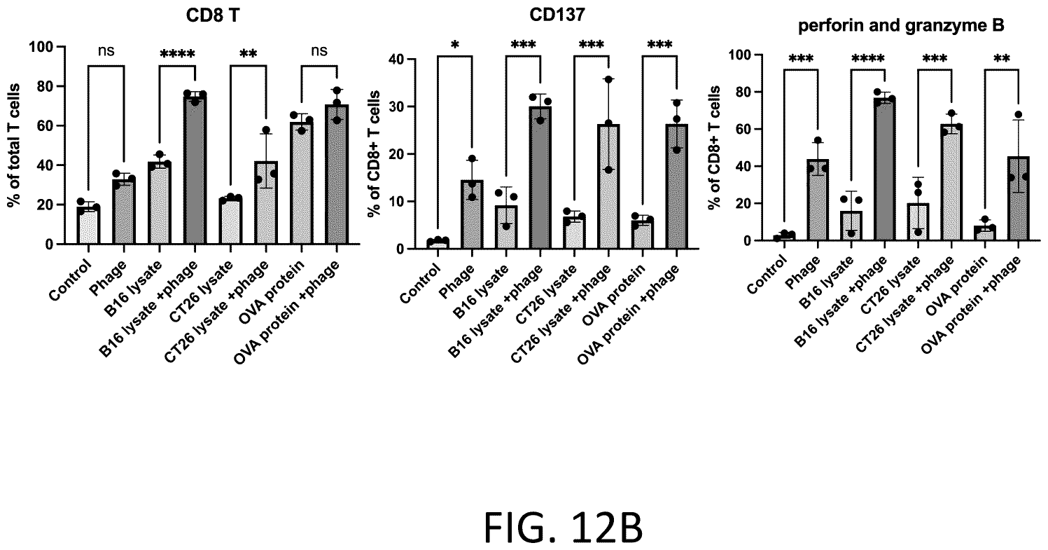

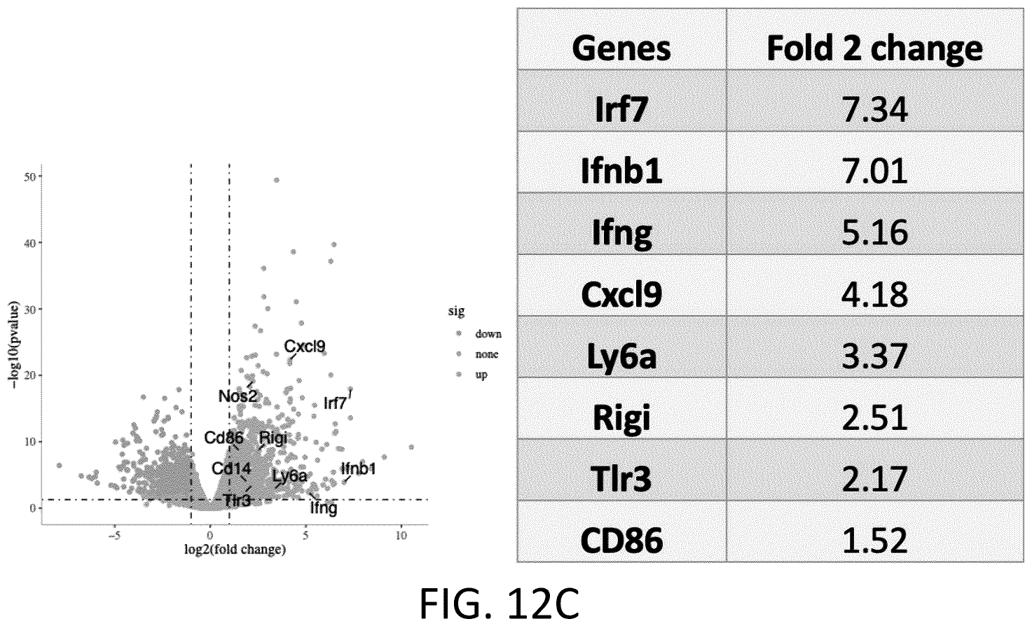

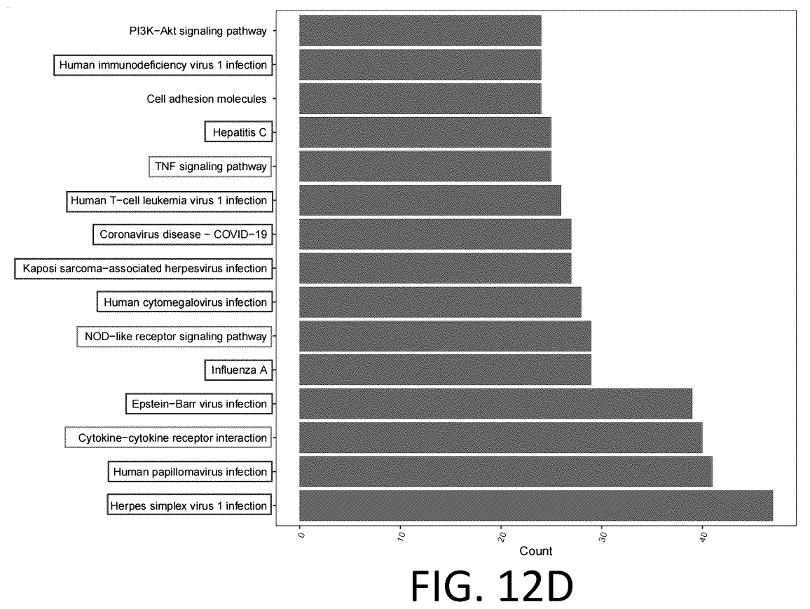

Further, to test if these dendritic cells have higher functionality in activating and guiding T cell actions, naïve T cells are incubated with phage-induced DCs together with the respective tumour antigens. T cells are observed to be skewed to a more cytotoxic phenotype after co-cultured with SCP induced DCs and demonstrated higher activation level and functionality based on the expression of cytotoxic effector proteins like perforin and granzyme B, suggesting that the DCs are not only activated, but have a higher ability to induce effector cellular activities as well (FIGS. 12A and 12B).

Dendritic cells co-cultured with SCP vectors were also found to have upregulated expression of multiple pro-inflammatory genes like IFN beta and gamma. Several key immune modulators like IRF7 and rig i were also found to be upregulated. DCs were also found to give similar response to SCP as to eukaryotic viruses like human immunodeficiency virus (HIV), hepatitis C and Influenza A infection (FIGS. 12C and 12D).

### Example 2: Presence of Both Antigens and DC-Targeting Peptides are Essential for Optimal Anti-Cancer Immunity

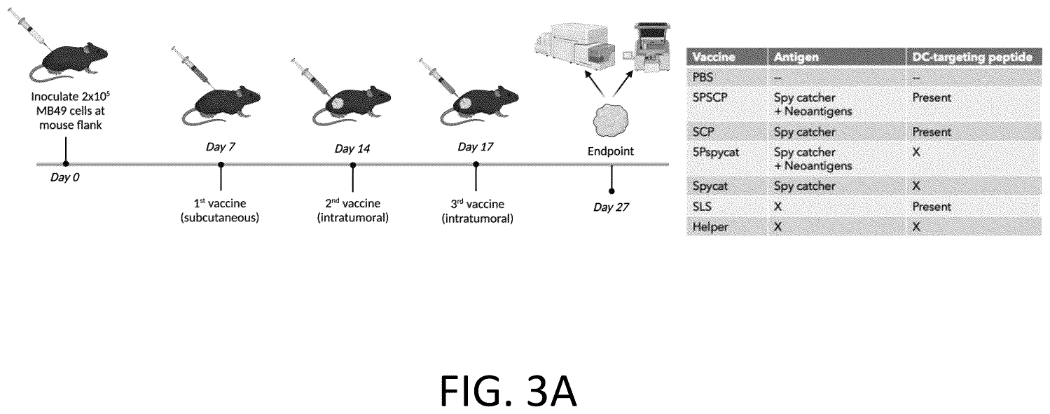

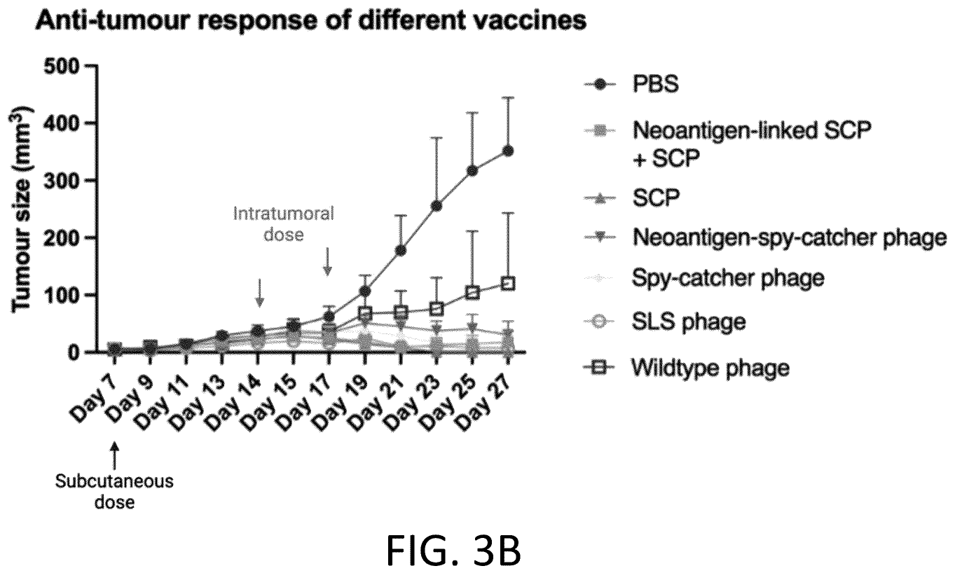

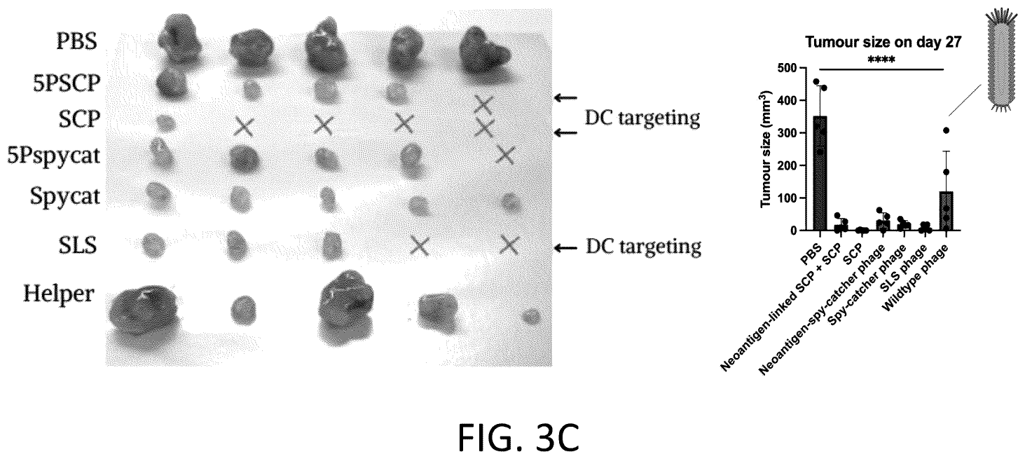

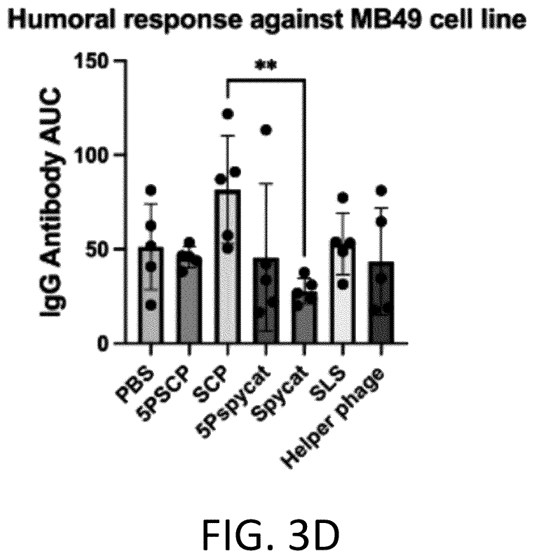

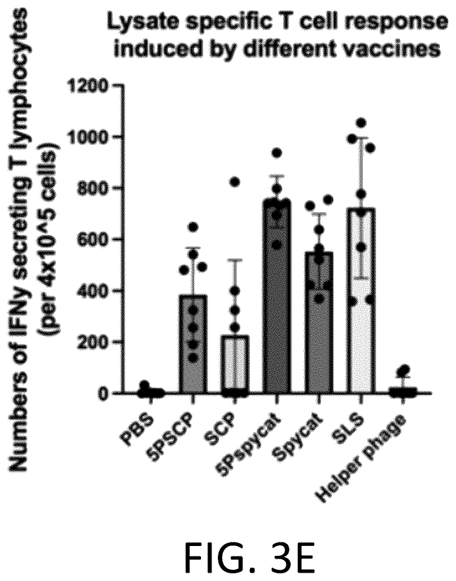

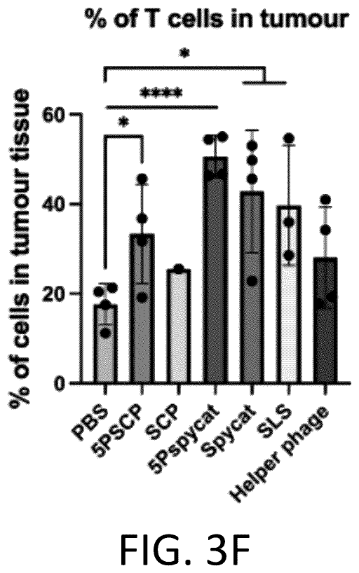

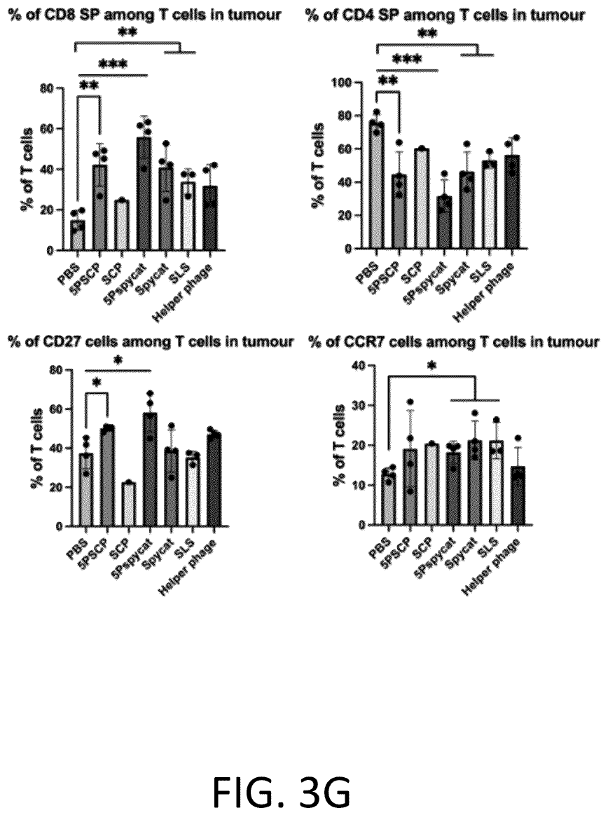

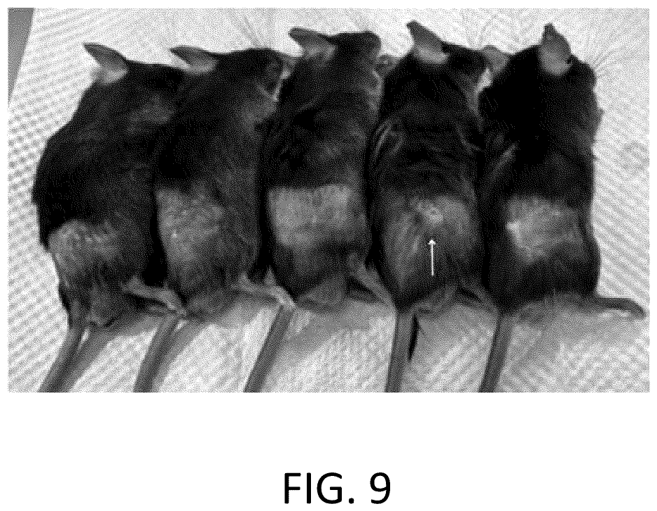

To look for the most important functional component of the neoantigen-SCP vaccine, vaccines are created with different combinations of protein expression. These vaccines are then tested with MB49 model (FIG. 3A). The vaccine composition for each dose is listed in Table 2 below. While mice treated with helper phage also showed significant tumour regression by around 3 times when compared to PBS group (p=0.01), groups treated with antigen and SLS-expressing phages had a much smaller tumour size at the end point (FIG. 3B). Meanwhile, SCP group showed the best anti-tumour effect, as 4 out of 5 mice had complete tumour regression (FIG. 9).

TABLE 2

| | Administration content and routes | | |
|---|---|---|---|
| Treatment | Day 7 | Day 14 | Day 17 |
| PBS | PBS (sc) | PBS (it) | PBS (it) |
| 5PSCP | 5 spy-tagged MB49 | SCP vector (without | SCP vector (it) |
| | neoantigen peptides | neoantigens) (it) | |
| | attached to the SCP | | |
| | vector (‘5PSCP’) | | |
| | with polyIC (sc) | | |
| SCP | SCP vector with | SCP vector (it) | SCP vector (it) |
| | polyIC (sc) | | |
| 5Pspycat | 5 spy-tagged MB49 | Spy-catcher | Spycat (it) |
| | neoantigen peptides | expressing phage | |
| | attached to the spy- | (‘spycat’) (it) | |
| | catcher expressing | | |
| | phage (‘spycat’) | | |
| | with polyIC (sc) | | |
| Spycat | Spy-catcher | Spycat (it) | Spycat (it) |
| | expressing phage | | |
| | (‘spycat’) (sc) | | |
| SLS | SLS-expressing | SLS (it) | SLS (it) |
| | phage with polyIC | | |
| | (‘SLS’) (sc) | | |
| Helper | MI3 helper phage | Helper (it) | Helper (it) |
| | (‘helper’) (sc) | | |

SLS and spy catchers are the two major additional elements on SCP when compared to helper phage. SLS targets DCs, while spy catchers can serve as a foreign antigen to mouse and elicit immune response. Among these two factors, DC-targeting appeared to play a more significant role in triggering anti-cancer immunity as mice in groups treated with the SLS component showed more remarkable repression in tumour growth (FIG. 3C). Tumours in SLS group (with an average tumour size of 7.91 mm³) are more than 15 times smaller than tumours in mice treated by helper phage (120 mm³), while 5PSCP group (17.6 mm³) performed around 1.7 times better than 5Pspycat group (30.6 mm³).

SCP showed a better treatment efficacy when administered without neoantigens in the first dose. It is hypothesized that neoantigens, as cancer antigens, may be less foreign to the host than spy-catcher proteins. This may mask the immunogenicity of SCP vectors and hence hamper the anti-tumour effect. Nevertheless, this experiment demonstrated that the presence of both the foreign antigens, be it spy catchers or neoantigens, and DC targeting peptides are crucial to achieve optimal treatment effects.

SCP also induced more robust cancer-specific humoral immunity than groups that express other protein combinations (FIG. 3D). The anti-MB49 antibody level is around 3 times higher in SCP group when compared to mice treated with phages that only express spy-catchers (p=0.0036). Administration of phage vaccines could also induce systemic cell-mediated anti-cancer immunity (FIG. 3E).

In the local tumour environment, T cell population significantly expanded in groups treated with neoantigen and SLS-expressing phages (FIG. 3F). Among the infiltrated T cells, the percentage of CD8+, CD27+ and CCR7+ cells increased when compared to PBS group (FIG. 3G). However, percentage of CD4+ T cells decreased in all treatment groups.

However, it is important to note that immune responses could decrease and return to the base level after the tumours are resolved. The systemic and local immunity measured in the SCP group therefore may not reflect the host response at its strongest action, as tumours in SCP-treated group resolved the earliest. Timepoint experiments are subsequently performed and examined how the immune microenvironment evolved throughout the treatment.

### Example 3: SCP-dependent anti-tumour immunity evolving with time

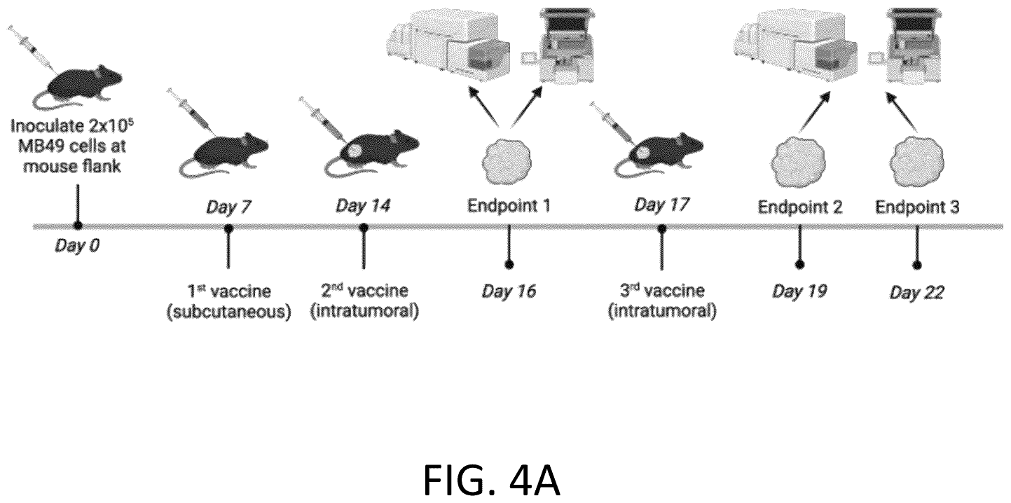

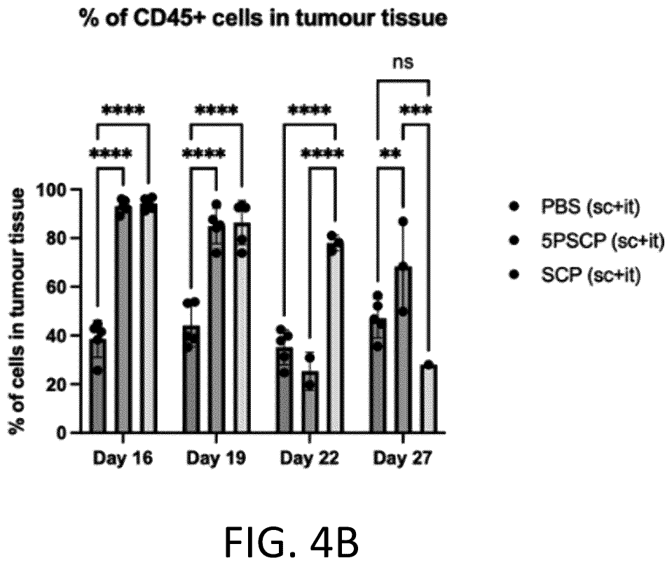

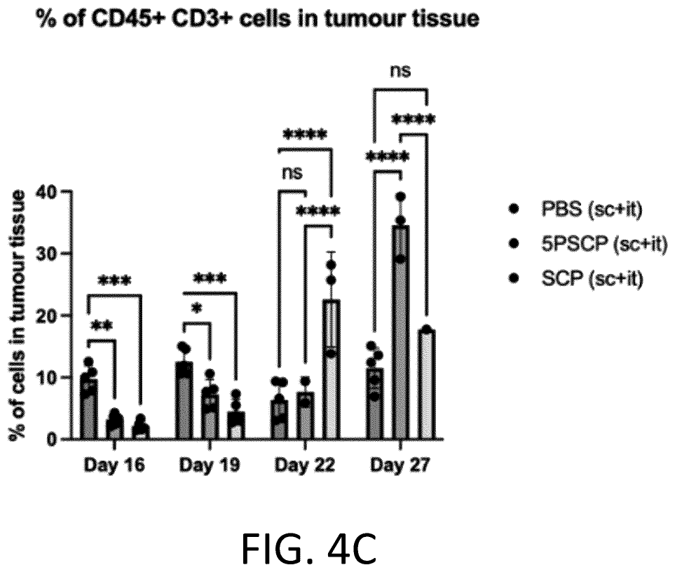

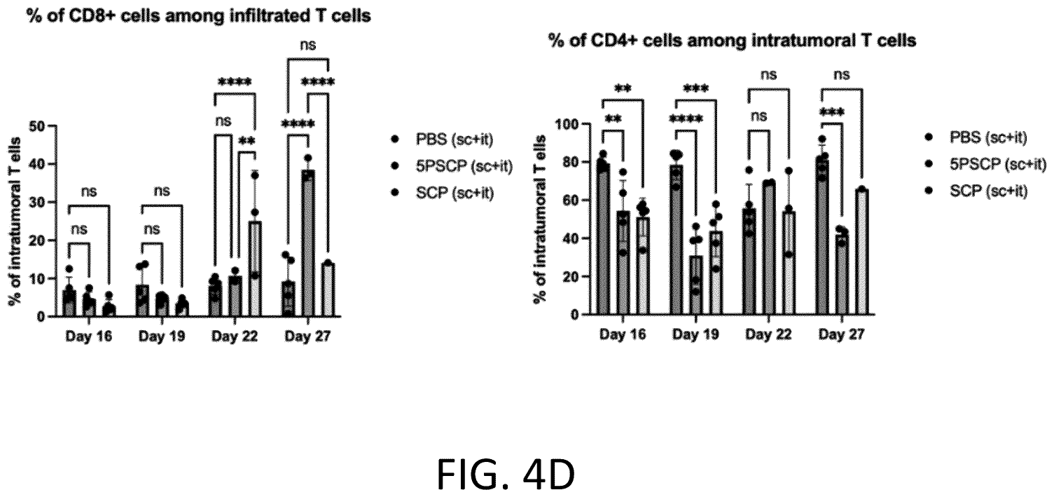

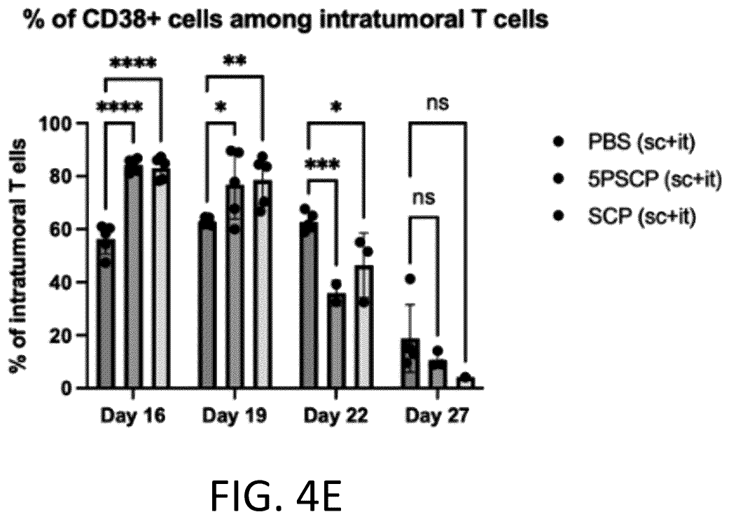

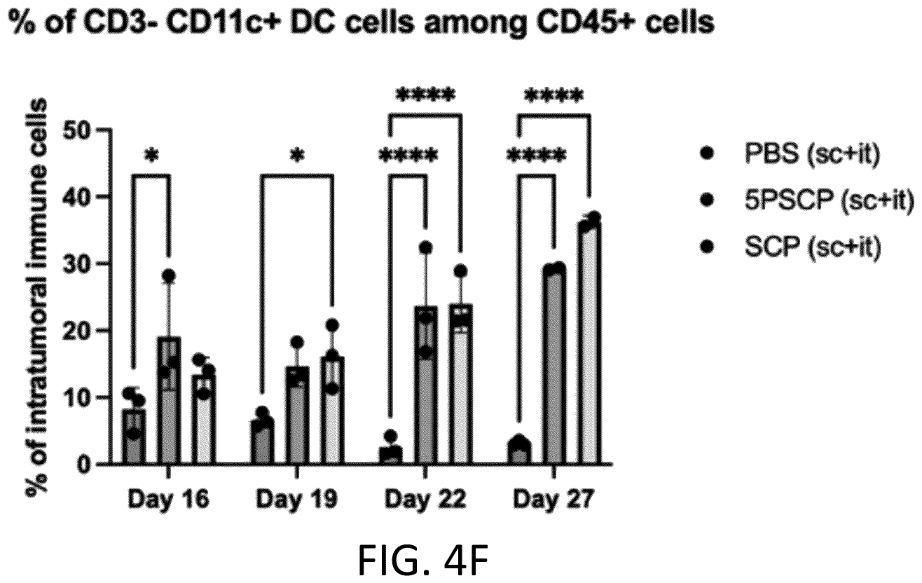

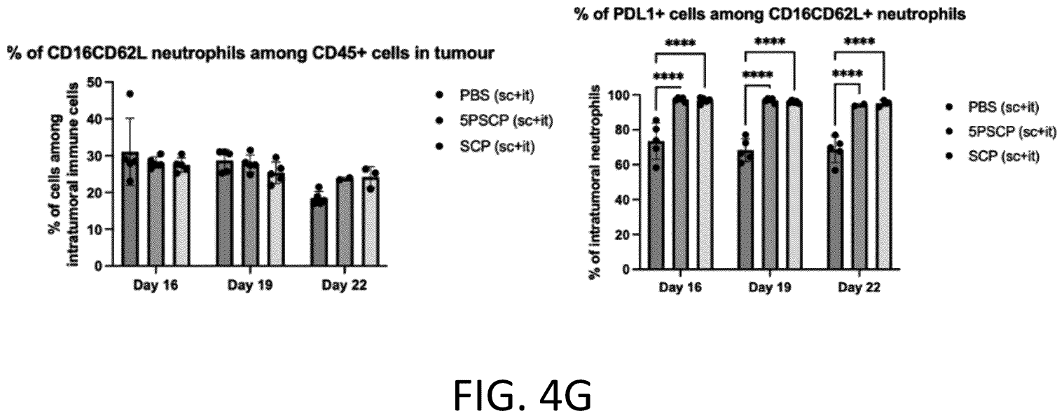

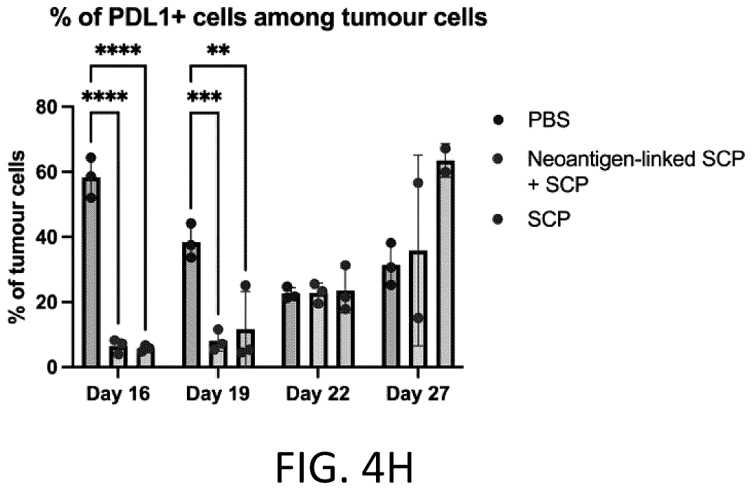

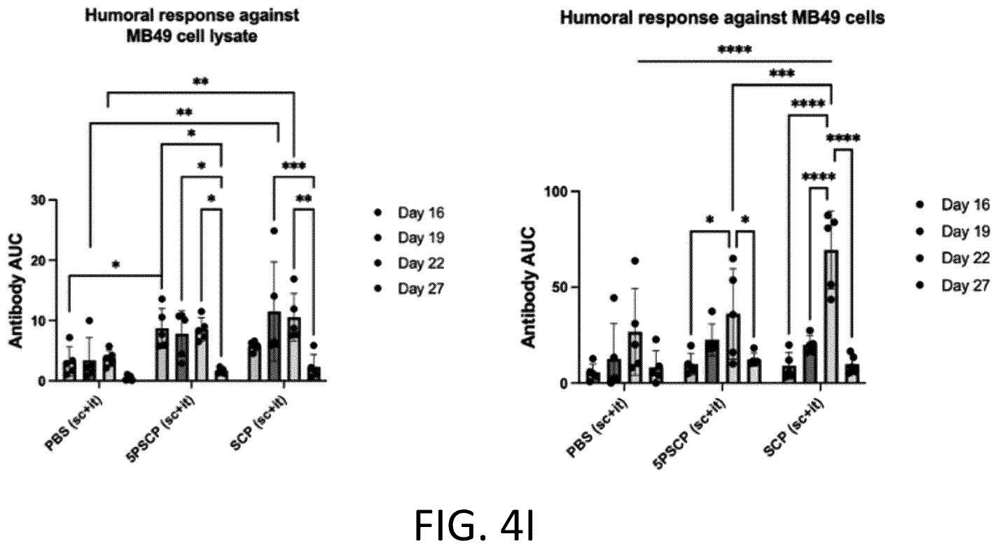

MB49 bladder cancer cells are inoculated in healthy C57BL6 mice as before. Mice are sacrificed on days 16, 19, 22 for evaluation of changes in tumour infiltration before the previous endpoint on day 27 (FIG. 4A).

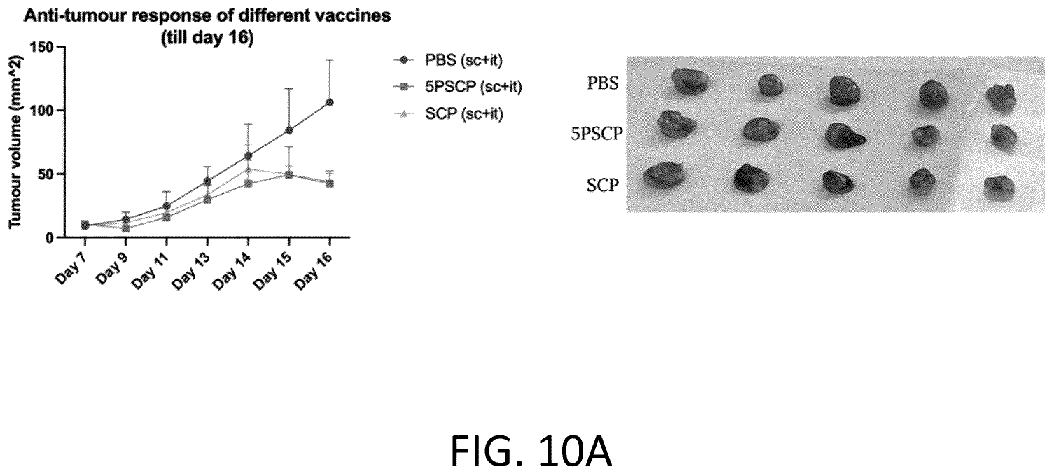

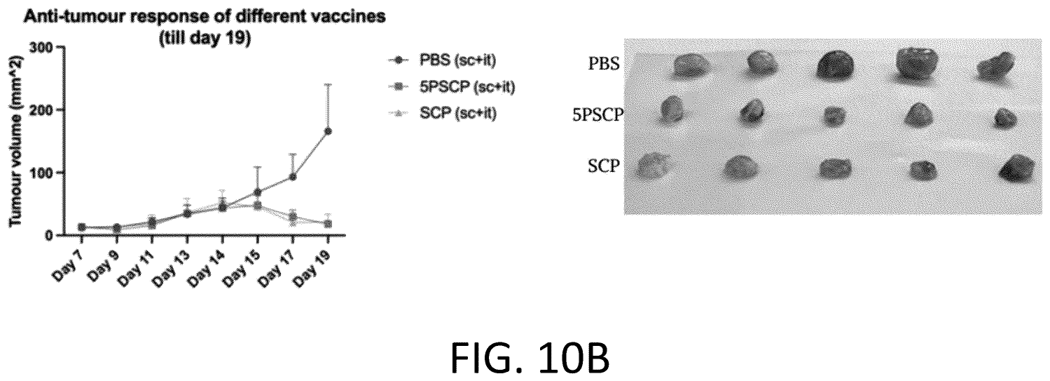

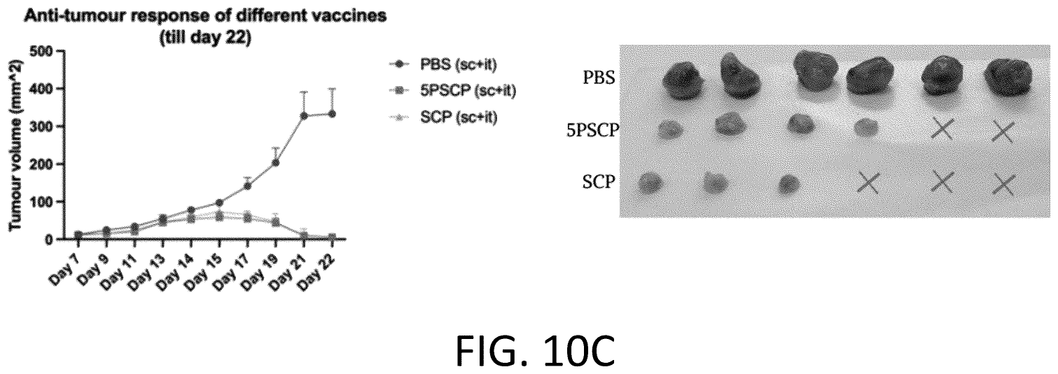

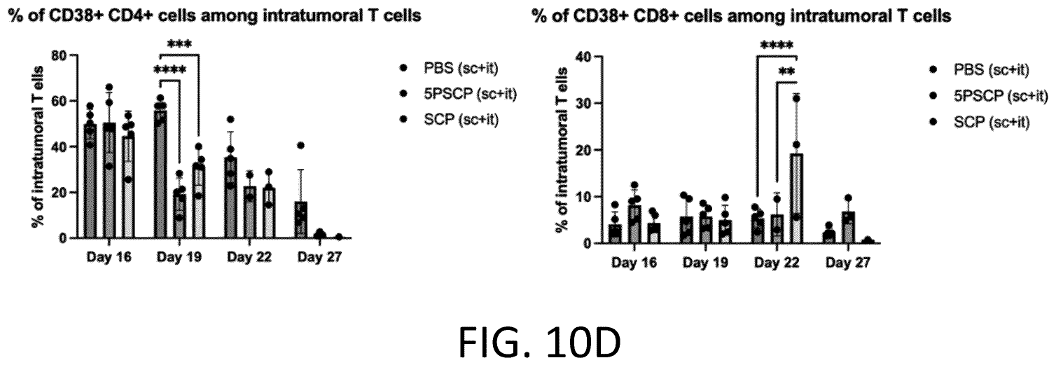

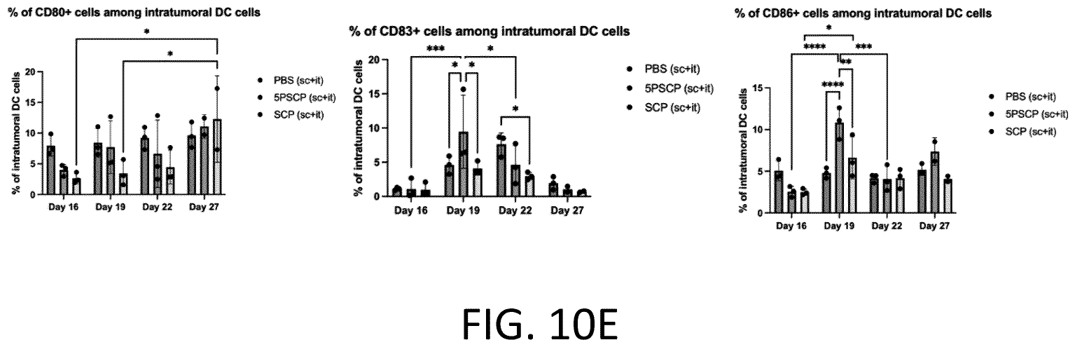

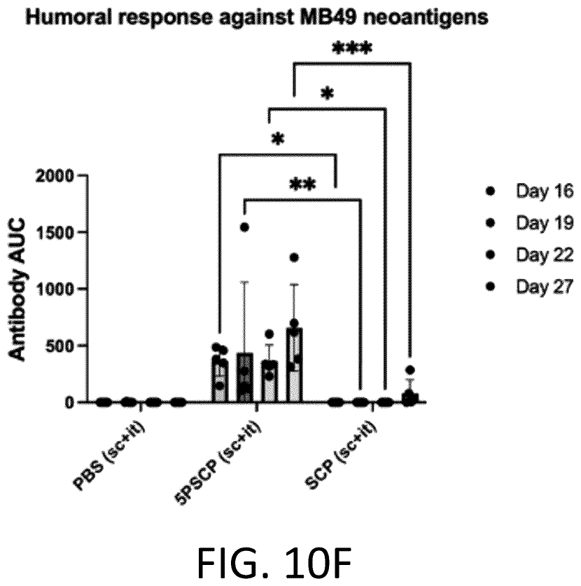

Tumour growth is not significantly restricted by the vaccine treatments in the early stage (FIGS. 10A to 10C). Yet, CD45+ cells are found to be the major population in tumour mass of the treated groups, with 93.1% of cells being CD45 positive in 5PSCP group and 94.1% in SCP group on day 16, 2 days after the first intratumoral dose (FIG. 4B). The CD45 population then gradually decreased with time after the last injection, suggesting that the local acute inflammatory response would revert back to normal with tumour resolution. Yet interestingly, majority of the infiltrated CD45 cells are not T cells (FIG. 4C). CD45+CD3+ T cells contributed to only around 2-7% of the infiltrated immune cells in the tumour mass of 5PSCP and SCP groups on days 16 and 19. The proportion of T cells within the tumour of the treated groups is also notably lower than that of the PBS group, which population remained stable at approximately 10% in the entire treatment. T cell percentage in the treatment groups did not increase and surpass that of the PBS group until around day 22. Nevertheless, when the tumours resolved, T cell population decreased again.

Among the intratumoral T cells, CD4+ cells are more abundant than CD8+ cells (FIG. 4d). CD8+ T cells had a similar progression pattern as total T cells, as they mainly expanded in population in the later stage on days 22 and 27. Yet interestingly, CD4+ T cells in the treated groups are significantly less than that in PBS group throughout the entire treatment. The drop is especially significant during the early stage of treatment by around 1.5 folds (p≤ 0.005).

On the other hand, CD38+ T cell population markedly increased by 1.5 times in the treatment groups on day 16 (p<0.0001) (FIG. 4E). Among these activated immune cells however, CD8+ T cells took up around 5% of all CD38+ cells and did not increase until around day 22 (Supplementary FIG. 4d). Similar to the number of infiltrated CD4+ T cells, the activated CD4+ T cell count also decreased in both treatment groups, with the most significant change of around 2 folds on day 19 (p≤0.0006).

The CD3-CD11c+ DC population expanded since the early stage of intratumoral phage treatment, and remained as a one of the major intratumoral immune populations at the later stage (FIG. 4F). The DC population is around twice as much as that of the PBS group on days 16 and 19 (p≤0.04), which then expanded to approximately 10 times on days 22 and 27 (p<0.0001). This population expansion maintained for a longer period than T cells, even after the tumours began to resolve. On the other hand, while CD80 expression on the DC cells increased over time with phage treatment (FIG. 10E), CD83 and CD86 expression levels peaked on around day 19.

Meanwhile, population of CD16CD62L neutrophils do not change with treatment (FIG. 4G). However, a significantly higher proportion of the neutrophils are activated by SCP vaccine, as around 96% of neutrophils expressed PDL1 in treatment groups. This demonstrated that SCP not only attracted infiltration of T cells, but also activated intratumoral neutrophils for anti-cancer actions.

Surprisingly, intratumoral injection of SCP also weakened tumour defence mechanism by remarkably reducing PDL1 expression on MB49 cells (FIG. 4H). PDL1 expression on tumour cells dropped drastically by 11 folds in treatment groups by day 19 (p<0.0001). Yet, flow cytometry results on day 22 showed that PDL1 expression on the tumour cells increased with time after the termination of SCP injection. Nevertheless, this result suggested that SCP vaccines could modify tumour expression patterns and weaken their defence mechanisms against host immune response and treatments.

Similar to the previous result, SCP vaccine, with or without neoantigen, elicited systemic humoral response against MB49 cell lysate (FIG. 4I). The anti-cancer antibody level increased from around day 16 and peaked on around day 22. After tumours resolved, the humoral response dropped significantly. Meanwhile, the antibody levels in PBS groups had no statistically significant change throughout the entire timeframe. Notably, different tumour antigens are targeted by the SCP vaccine when administered with and without neoantigens (FIG. 10F). Nevertheless, the total humoral response is stronger in the SCP group, when mice are not exposed to the chosen neoantigen peptides.

Overall, it is demonstrated that SCP vaccine, with or without neoantigens, can induce multi-factorial changes in tumour microenvironment by attracting and activating anti-tumour immunity and reducing tumour evasiveness to immune attacks.

### Example 4: Extended Use of SCP Vectors

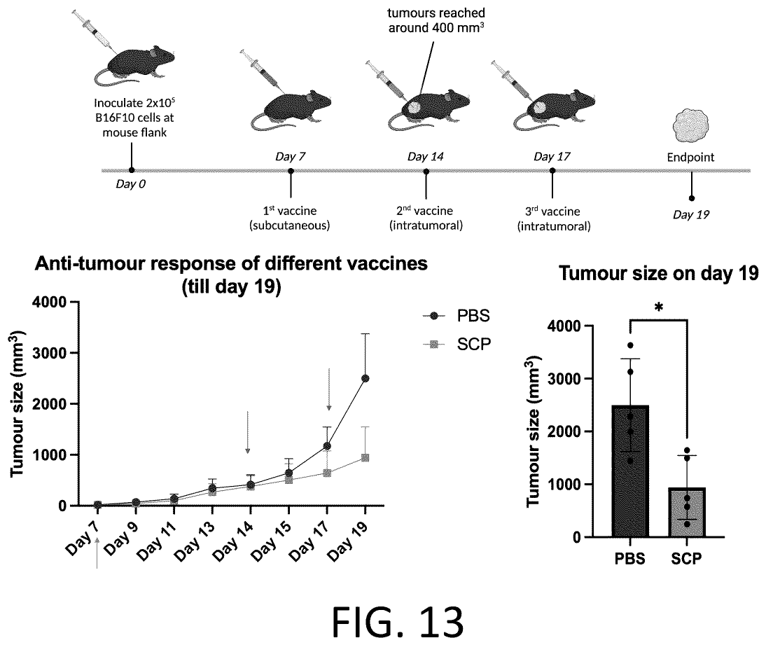

SCP vectors are also tested on whether they are applicable in other tumour models with more aggressive growth and if the treatment can induce long-term protection. SCP is intratumorally administered to B16F10 melanoma tumours when they reached sizes of 400 mm³. It is found that SCP could control the aggressive tumour growth even when the tumours reached a certain size (FIG. 13).

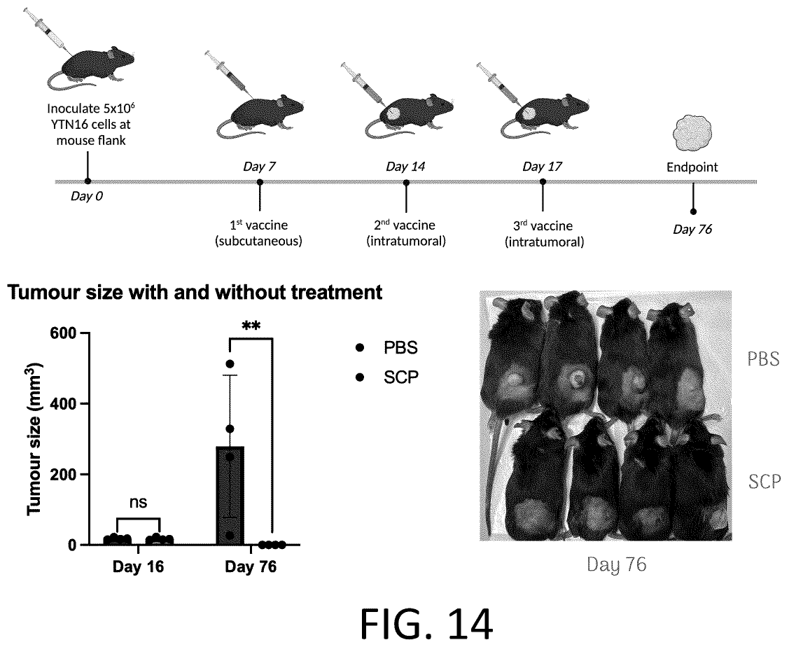

SCP is also tested on YTN16 gastric cancer model, and found that the 2 intratumoral doses on days 14 and 17 could elicit a long-term anti-tumour protection for more than 2 months (FIG. 14).

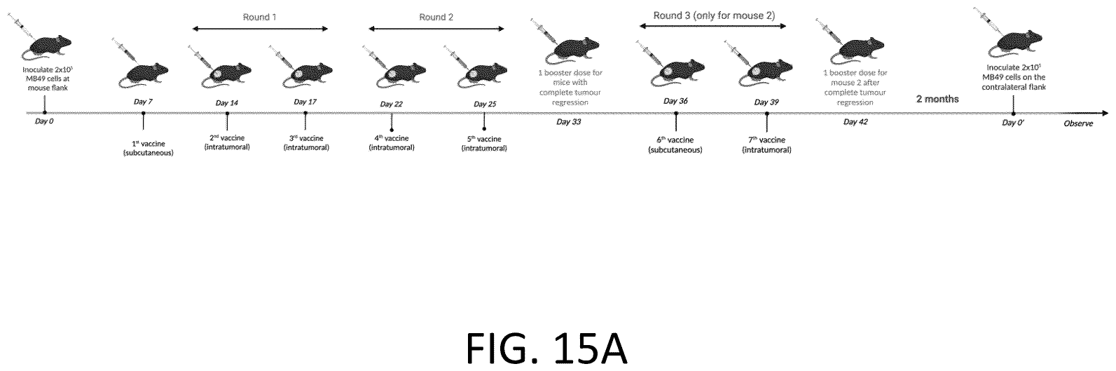

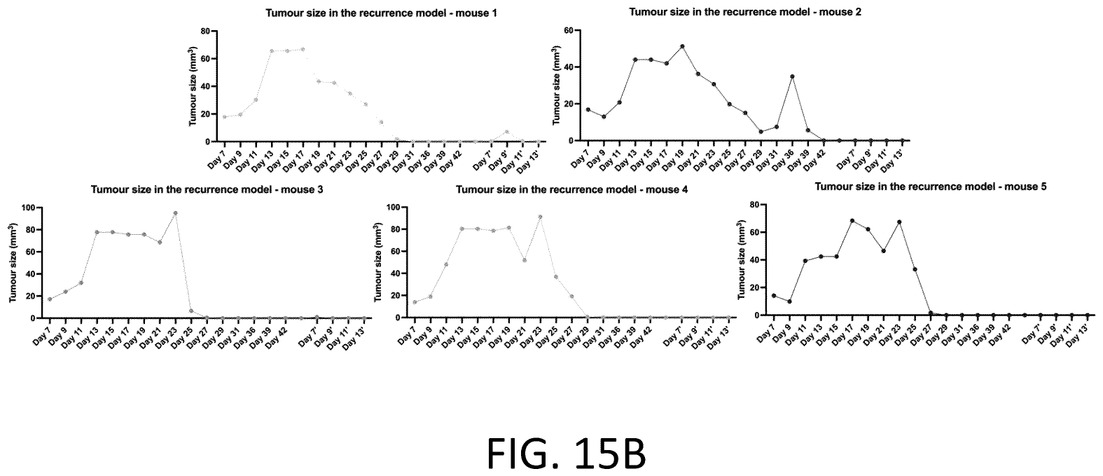

To verify if SCP treatment could induce long-term anti-tumour memory immunity, the regimen is refined to two intratumoral SCP injections per week until complete tumour resolution. MB49 cells are subsequently reseeded on the contralateral flank two months after achieving tumour regression. No tumour growth was observed in all mice after two weeks, with only two mice developing small bumps that regressed within 2-4 days, suggesting the induction of long-term anti-tumour memory immunity (FIGS. 15A and 15B).

The above results showed that SCP displayed effective anti-tumour effects, yet the effect is much more significant when administered intratumorally. A possible explanation is that phages can induce vigorous inflammation in the tumour environment. The active inflammation hence led to immunogenic tumour cell death for the release of tumour antigens, which immunogenicity is further amplified by the DC targeting phages as a foreign antigen.

### Example 5: Investigation of the Action Mechanism of the SCP Treatment

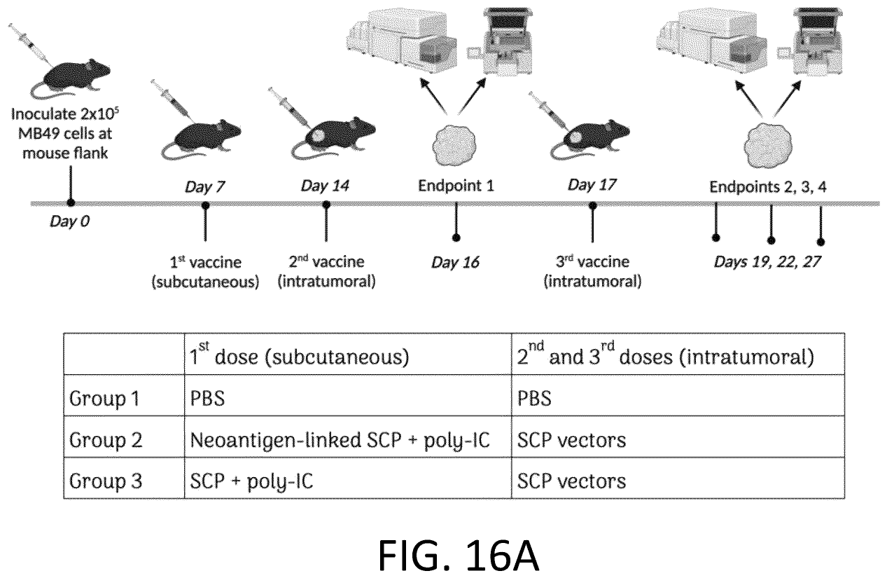

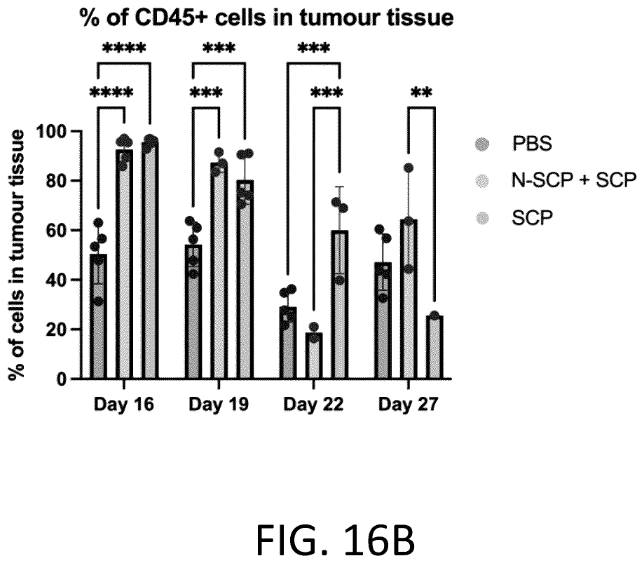

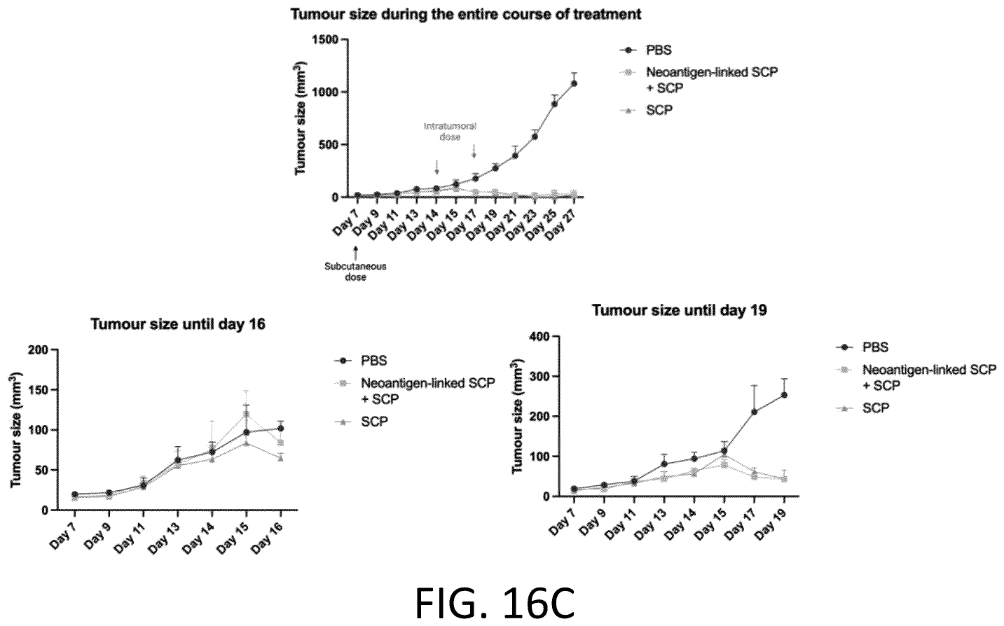

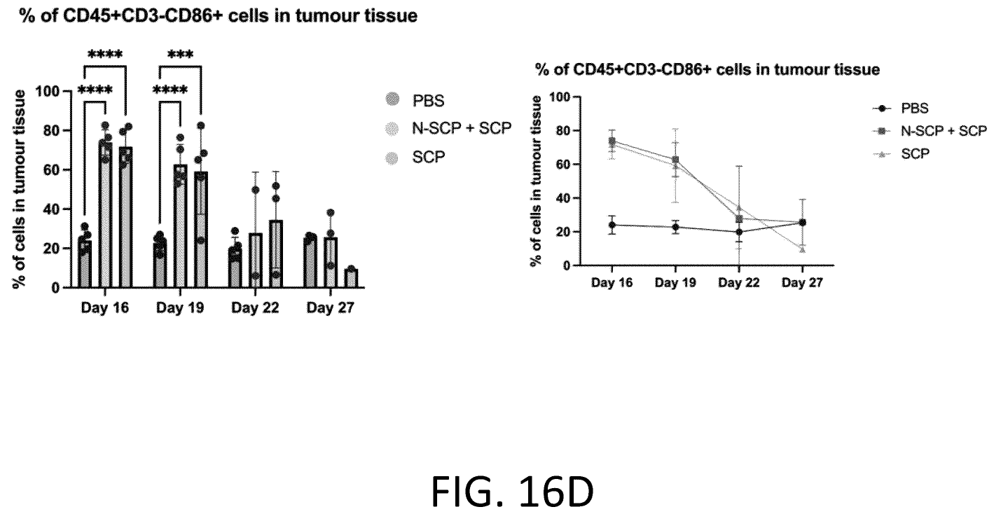

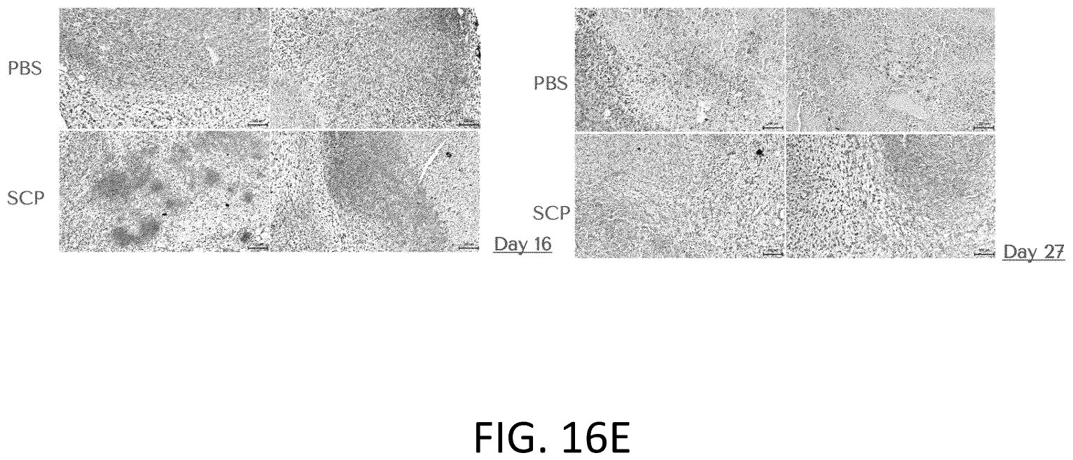

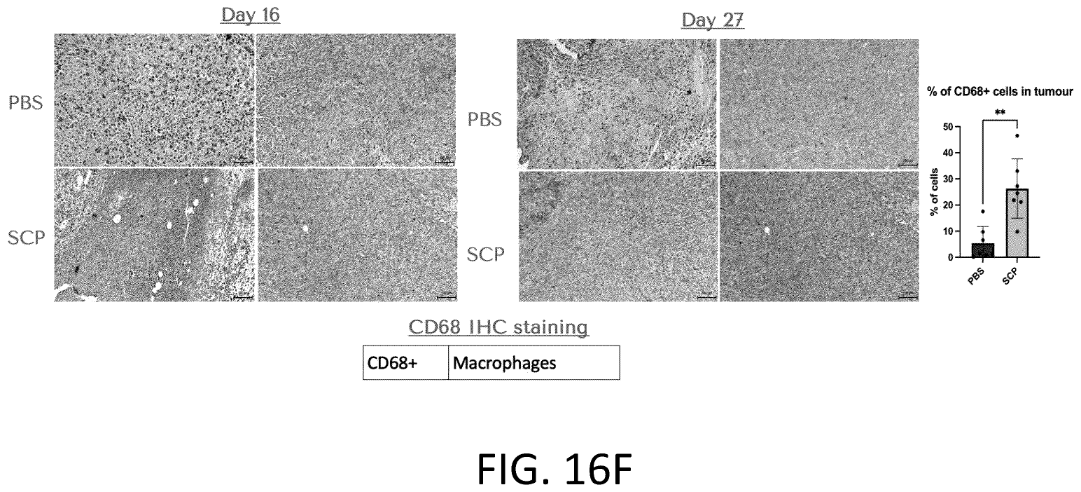

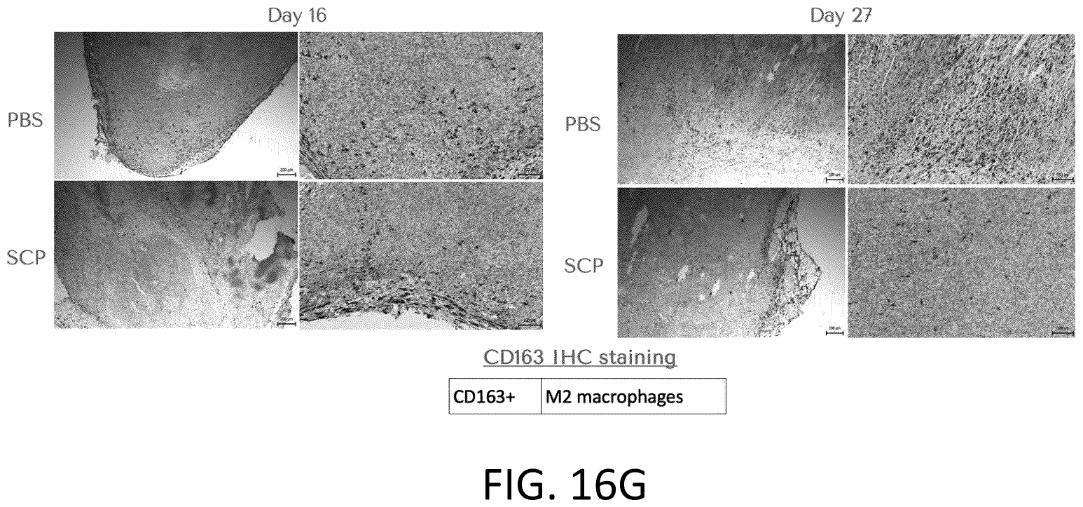

The tumours are first isolated and evaluated the immune environment along the course of treatment. While the tumour size was not restricted after the first dose of intratumoral treatment, over 90% of the intratumoral population were CD45+ immune cells on day 16. The percentage remained high at around 80% on day 19, and only gradually decreased on day 22 to a level comparable to the PBS control group. This is in line with the inflammatory presentation of the tumours observed, suggesting that immune infiltration was the major cause of ‘pseudo-progression’, or the initial increase in tumour size (FIGS. 16A to 16C).

Among the infiltrated immune cells, the CD86+ population expanded most significantly in the early stage of treatment. CD86 is known as a marker for type 1 macrophages and a co-stimulatory marker for various antigen presenting cells, including DCs, macrophages, and activated memory B cells. IHC staining is then performed to visualise the spatial distribution of the CD86 cells. With SCP treatment, the CD86+ population expanded initially on day 16 with diffuse infiltration and clustering observed. The cells later showed a similar peripheral distribution in both SCP- and PBS-treated tumours on day 27 (FIGS. 16D and 16E).

As CD86 is an established marker for M1 macrophages, we also stained CD68 to look at the distribution and infiltration of macrophages on days 16 and 27. The staining revealed that macrophages were more abundant in the SCP-treated tumours, and its population remained expanded as the tumours resolved on day 27 (FIG. 16F). On the other hand, while PBS-treated tumours showed increased CD163+ infiltration beyond the margins at the endpoint, SCP-treated tumours retained these cells predominantly at the periphery to the tumour margins (FIG. 16G). These staining revealed that SCP can increase the general infiltration of macrophages, while the CD86 phenotype mostly increase in the early stage of treatment and gradually decrease with tumour regression. The M2 phenotype on the other hand is also restricted at the tumour periphery with phage treatment, suggesting a more active phenotype with phage administration.

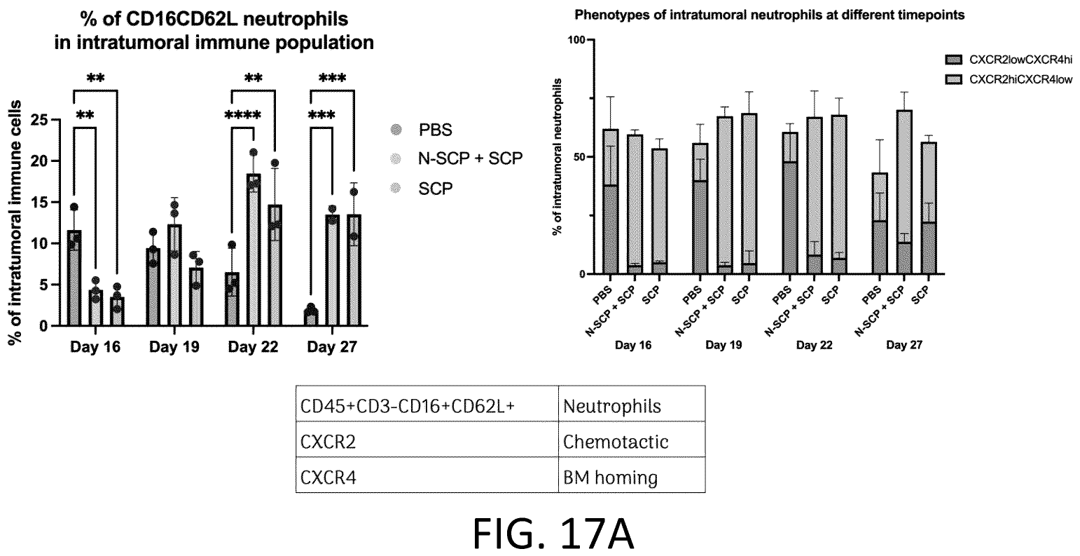

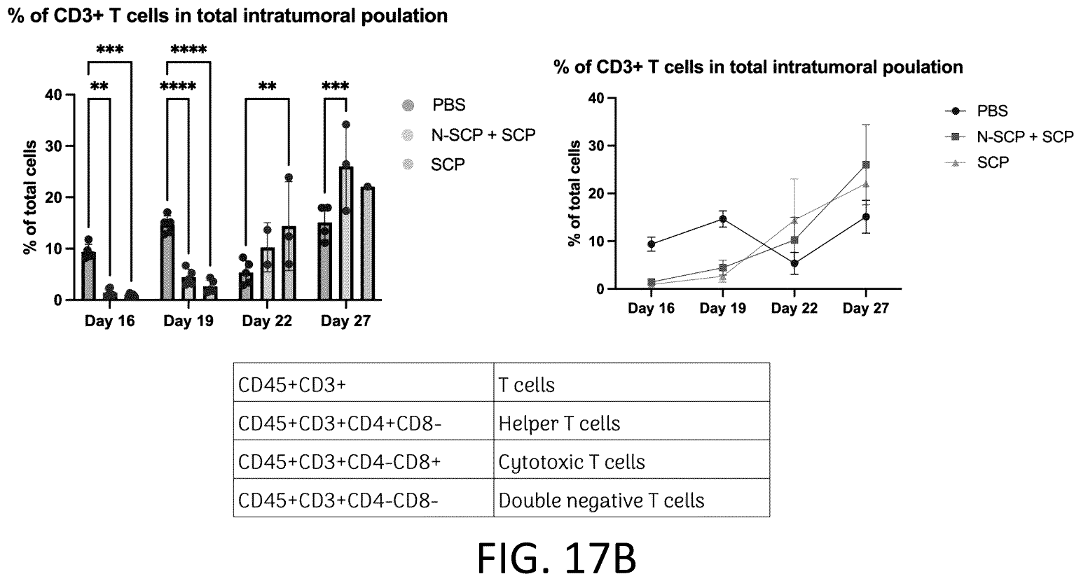

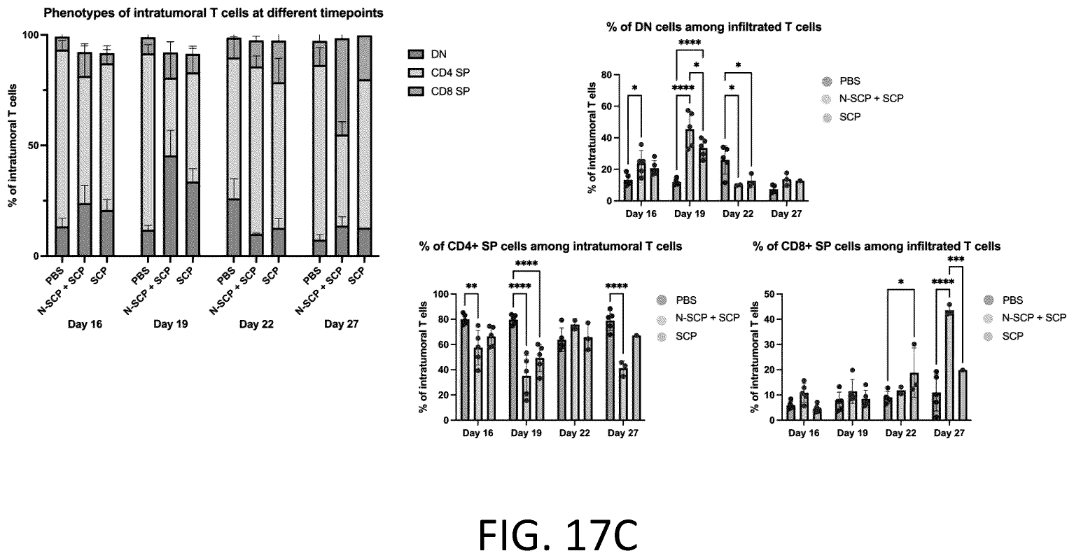

A phenotypic change is also observed for the neutrophil population. The overall population decreased initially and expanded around day 22. However, the cells consistently expressed higher levels of the chemotactic marker CXCR2 and lower levels of the bone marrow homing marker CXCR4 (FIG. 17A), indicating an inflammatory and active phenotype that may induce immunogenic cell death and the release of tumour antigens. There was also ample evidence that neutrophils played crucial roles in clearing aggregates of dead tumour cells that have undergone spontaneous apoptosis or therapy-induced apoptosis. The increase in neutrophil population in the later stage of SCP treatment may be related to the clearance of inflammatory debris after the acute local immune reactions.

Intratumoral T cell population showed similar trend as neutrophils, with an initial decrease and expanded from day 22 till the endpoint (FIGS. 17B and 17C). This aligns with the previous understanding that adaptive immunity comes into play at a later stage. Meanwhile, interestingly, the double negative (DN) T cell population expanded substantially with phage administration during the early stage of treatment, before shifting towards the cytotoxic phenotype towards the endpoint. Although DN T cells are mostly known as the immature T cells residing in the thymus, previous studies showed that DN T cell population can expand in inflamed tissues as well. These cells can also be pro-inflammatory and possess cytotoxicity towards tumour cells. The shift in T cell phenotypes, the causes and the indications would be a potential topic for further investigation to optimise anti-tumour immunity in the future.

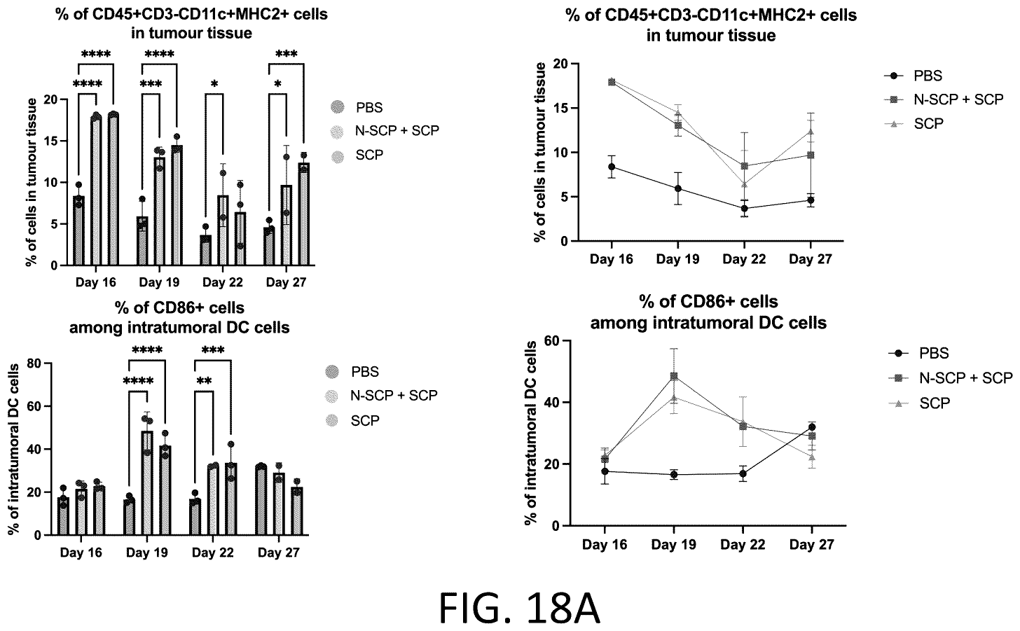

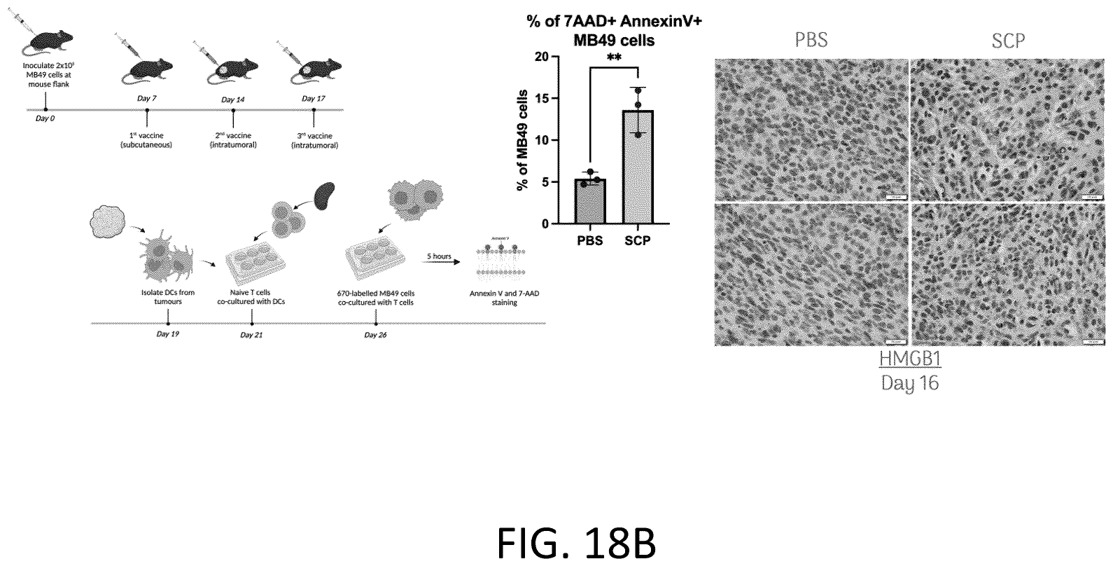

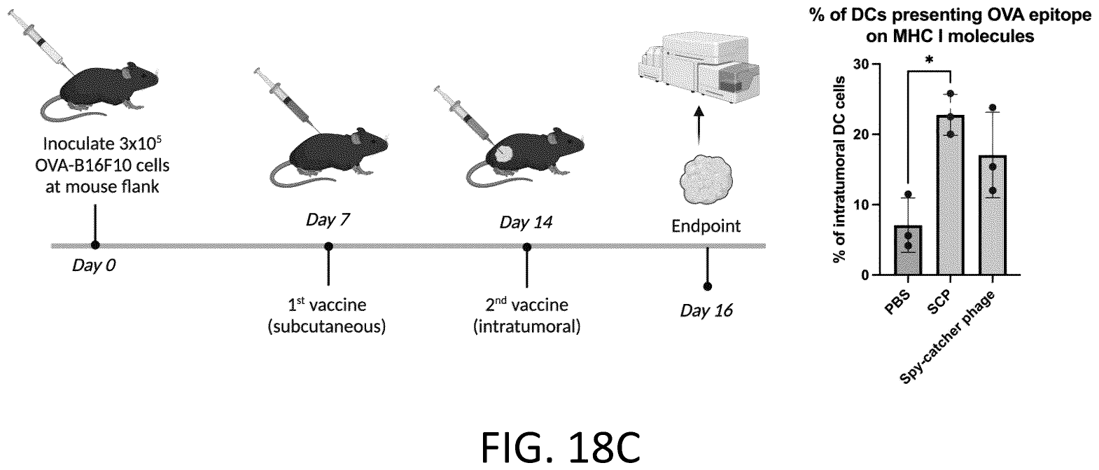

As SCP contains a DC-targeting component, the focus is shifted on dendritic cells. Intratumoral DCs population indeed expanded with treatment, also with a higher expression level of CD86. However, while the overall DC population remained expanded at the endpoint, most cells were not in an activated state by day 27, with CD86 levels comparable to the PBS group (FIG. 18A). This suggests a change in DC status between the initial active inflammatory phase and the later resolution phase.

The antigen-specific functionality of intratumoral dendritic cells (DCs) is further characterized by isolating them on day 19 and co-culturing with naïve T cells. T cells co-cultured with DCs isolated from treated tumours demonstrated significantly enhanced cytotoxic activities against MB49 cancer cells (FIG. 18B), confirming that SCP-treated DCs effectively engulfed MB49 antigens and educated T cells to target cancer cells.

The presence of tumour-specific immunity in the TME is further confirmed by HMGB1 staining (FIG. 18B), which is usually confined to the nucleus. (Yet, they can be shuttled to the cytoplasm or get secreted extracellularly under cell stress.) The IHC staining revealed a dispersed intracellular distribution and hollow nuclei in the treatment group, indicating the presence of cell damage after phage administration.

To further demonstrate that tumour antigens can be taken up by intratumoral dendritic cells, OVA proteins are used as a measurable antigen and performed the experiment in an OVA-expressing B16 melanoma model. The results showed a significant increase in the percentage of DCs presenting OVA MHCI epitopes in SCP-treated tumours (FIG. 18C), indicating enhanced DC ability to process and present tumour-associated antigens and elicit robust antigen-specific immunity.

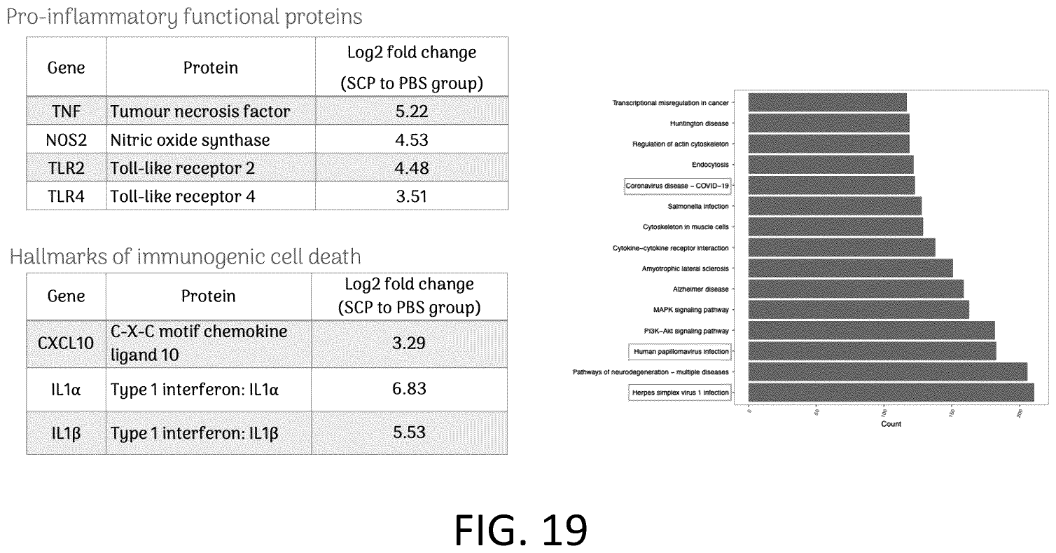

The RNA levels of multiple pro-inflammatory functional proteins and hallmarks of immunogenic cell death are also observed to increase with treatment (FIG. 19). The abundance of these factors provided proof of active and functional inflammation in the TME, which could create a stressful environment and lead to tumour cells damage. TLR2 and 4, which are the receptors responding to HMGB1 is also upregulated in the treated tumours. Similar to the in vitro experiment, anti-viral pathways are found to be upregulated in the tumour environment after SCP administration.

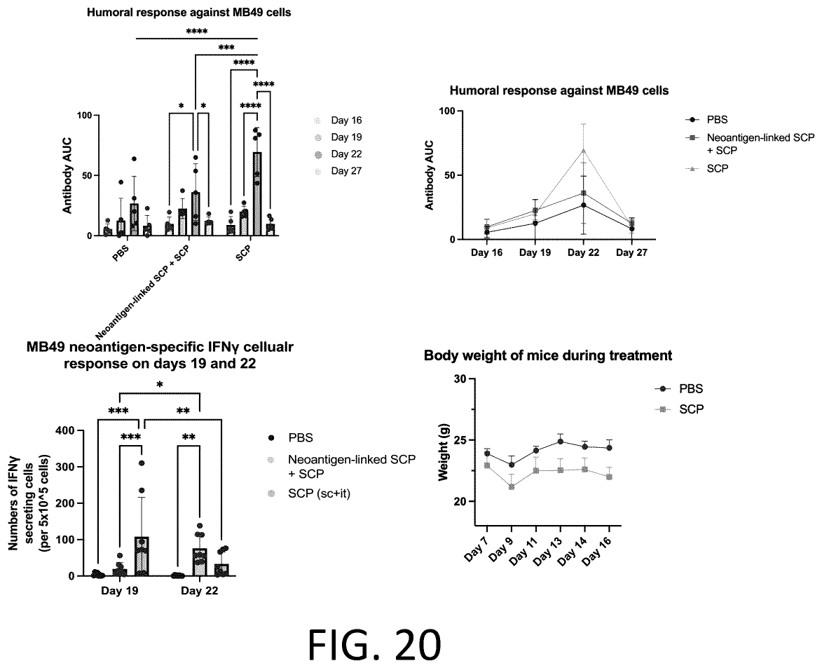

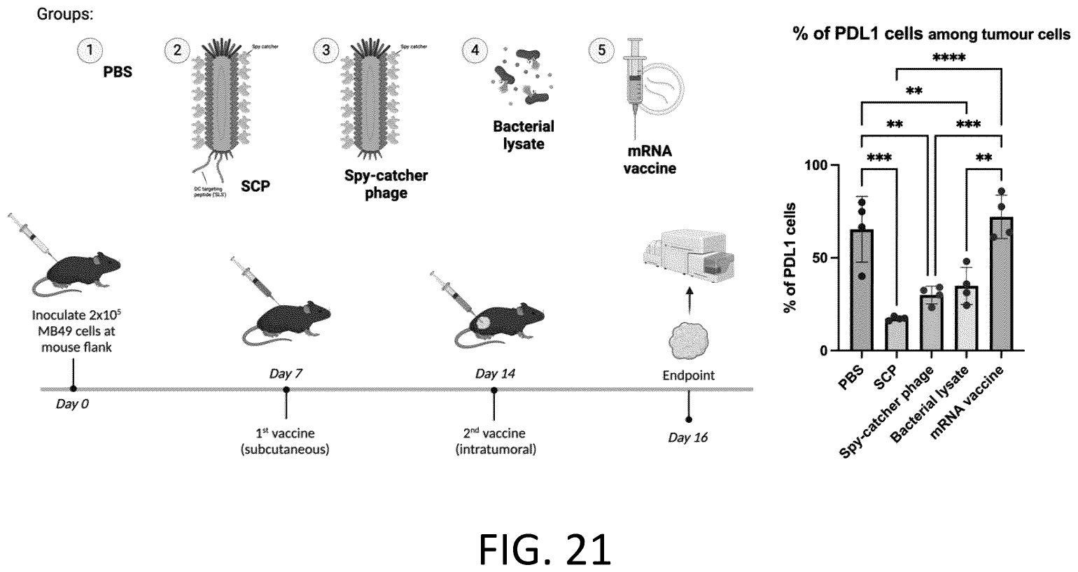

Other than local immune reactions, intratumoral SCP administration also induced systemic antigen-specific cell-mediated and humoral immunity that peaked on days 19 and 22 and subsided after tumours resolved. Notably, the weights of the mice also do not change during the treatment (FIG. 20), suggesting controlled systemic immune reaction and limited toxicity.

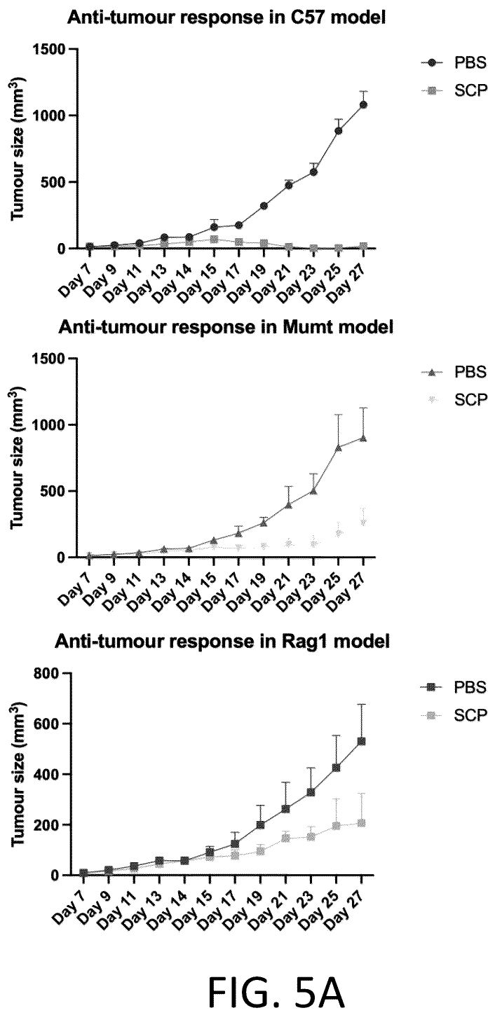

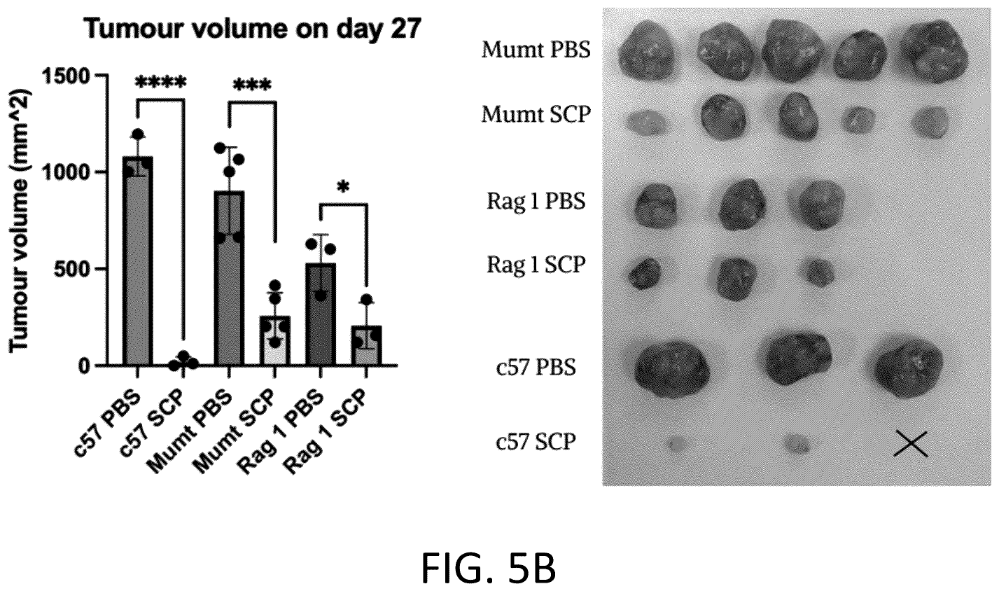

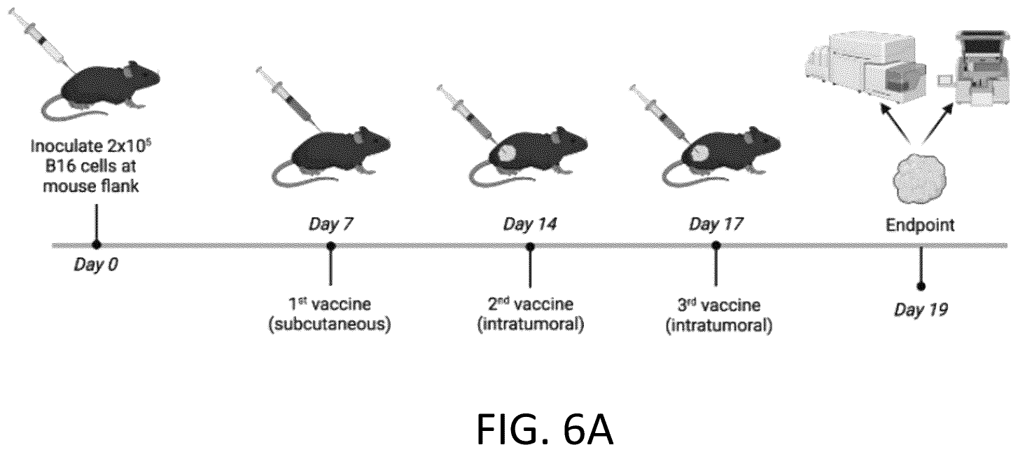

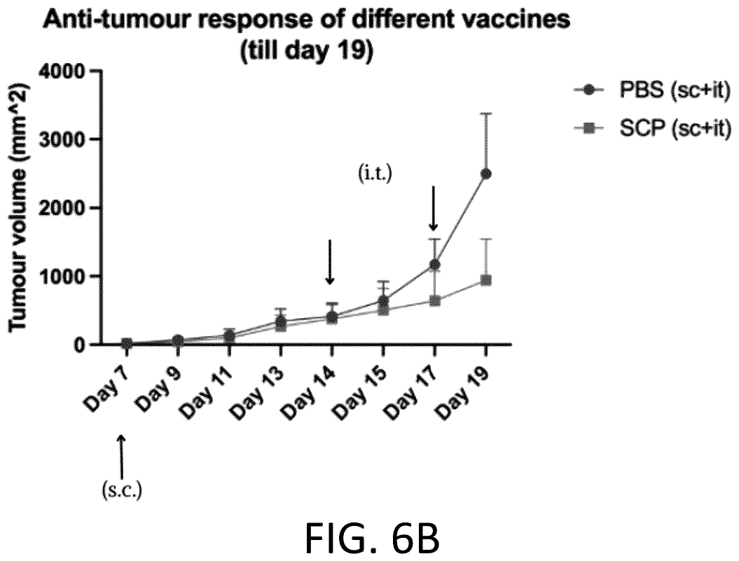

As previous data revealed that both innate and adaptive immunity are involved in the treatment effect, MB49 tumours are treated on Mumt and Rag 1 mice to evaluate the importance of B and T cells in SCP-based immunity. Without B cell actions, Mumt mice reduced the tumour size by around 3.5 folds (p=0.0005) (FIGS. 5A and 5B). Meanwhile, when B and T cell actions are both demolished, Rag 1 mice could still limit tumour growth by around 2.5 folds (p=0.0412) (FIGS. 5A and 5B). This therefore demonstrated that other than humoral and cell-mediated adaptive immunity, innate response is also essential for the anti-tumour effects of SCP.

It is then tested if SCP vaccine could control late-stage tumour growth with the more aggressive B16F10 model. C57BL6 mice are inoculated with B16F10 melanoma cells at the flank area. Mice are immunized with SCP vaccine subcutaneously on day 7, and the tumours are treated on days 14 and 17 (FIG. 5A). It is observed that although SCP reduced tumour size by 2.65 folds (FIG. 5A), the vaccine induced rather local effect on large tumours by causing tumour necrosis mainly at the injection site. It is hence deduced that a more even and diffuse administration method could improve anti-cancer efficiency.

Based on the above results, it is deduced that intratumoral administration of SCP reprograms the tumour microenvironment by inducing acute inflammation, with dendritic cells (DCs) and macrophages as initial key players, followed by T cell and neutrophil actions at later stages. Meanwhile, systemic B and T cell responses also helped with tumour resolution. These immune reactions create a robust inflammatory background, promoting immunogenic tumour cell death and activation of tumour-specific immune responses.

On the other hand, as SCP carries a dendritic-cell-targeting component, DC functions and activations could be heightened for a more robust immune response. The phage vector itself could also serve as a danger signal to boost tumour antigen immunogenicity.

### Example 6: SCP Modification of the Tumour Microenvironment from Multiple Perspectives

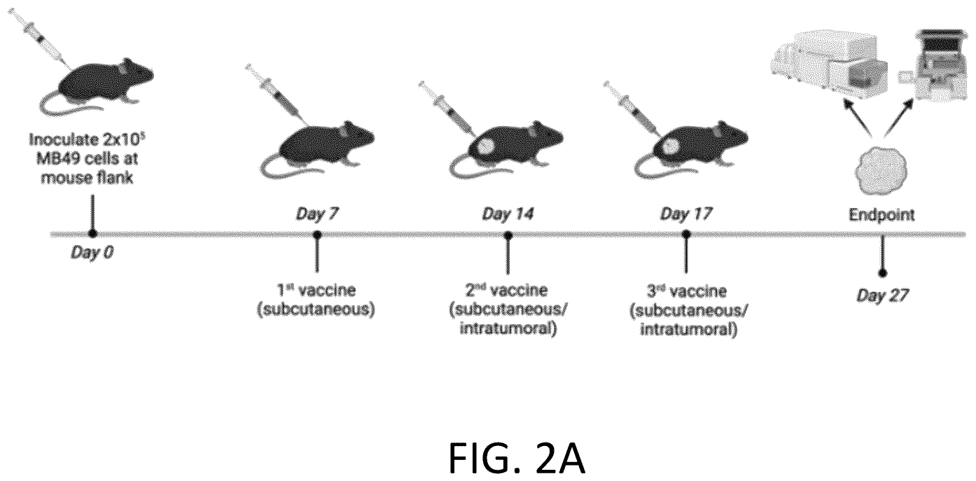

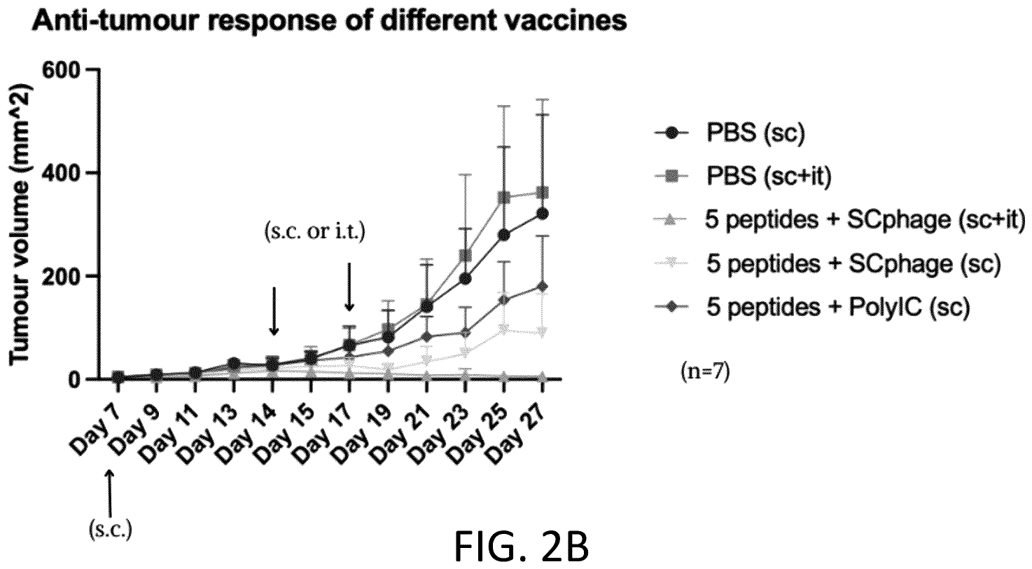

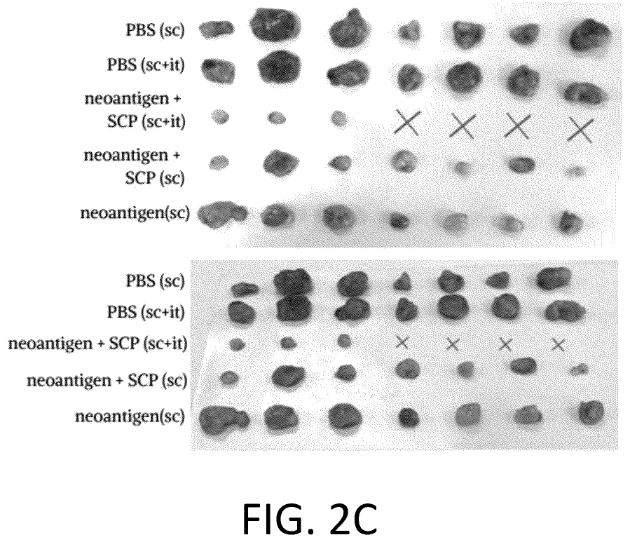

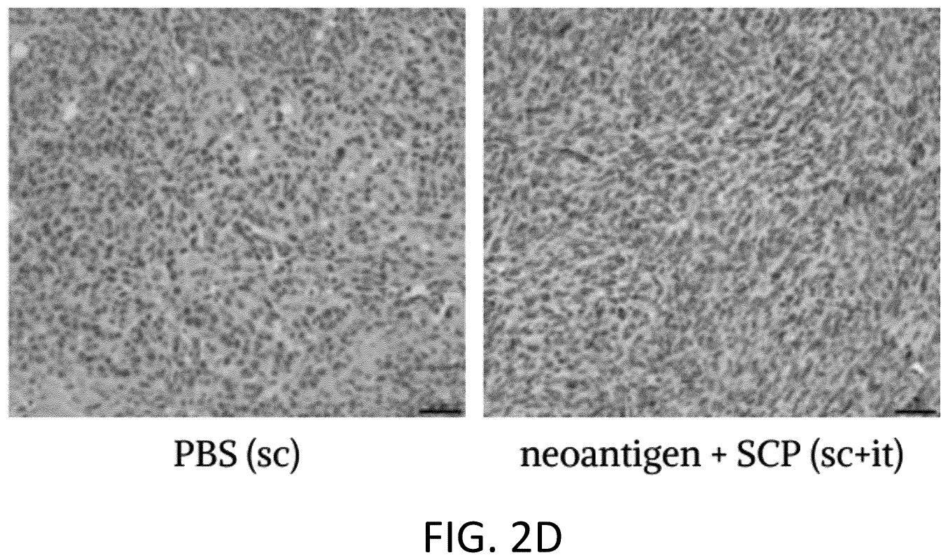

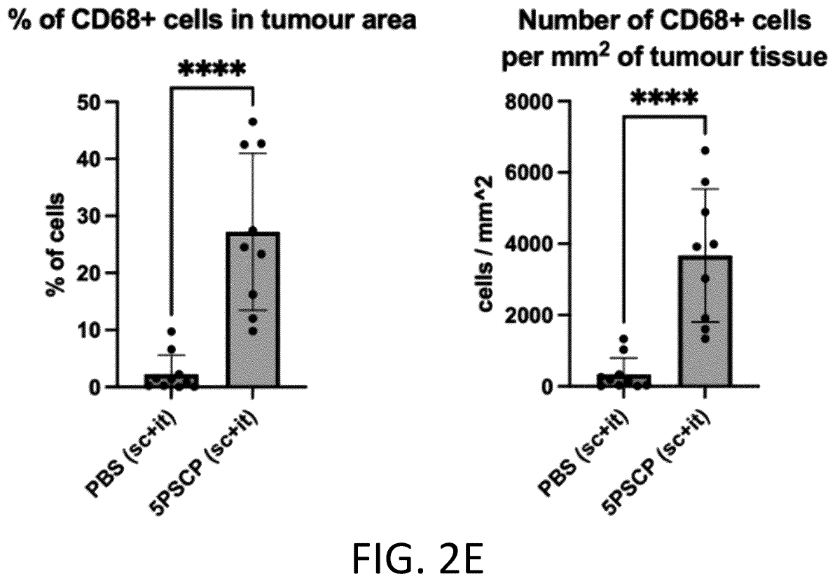

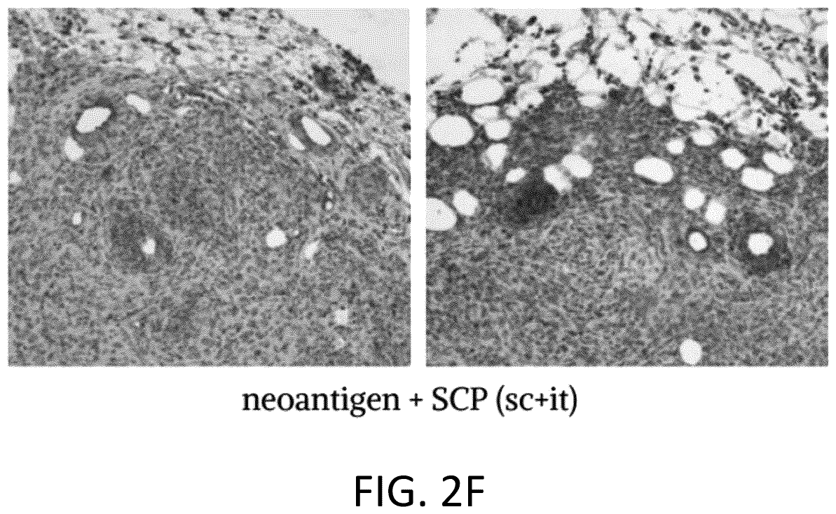

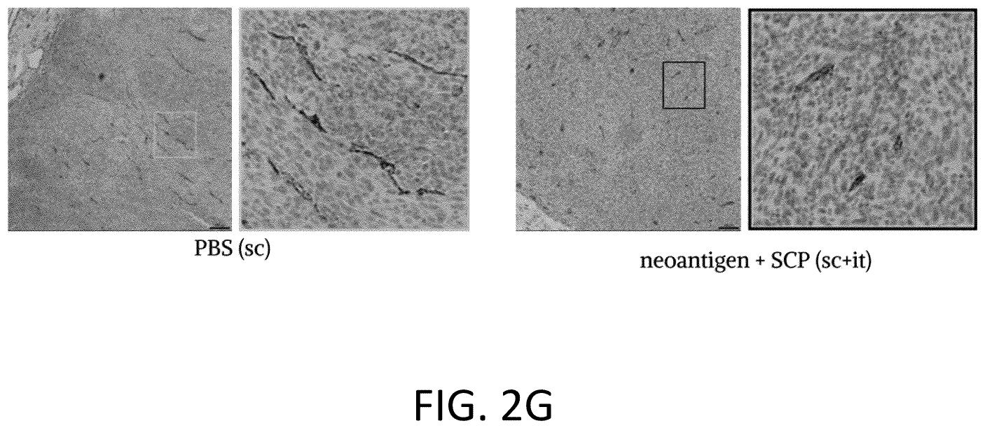

To examine the anti-tumour effects of SCP, in vivo studies are performed with MB49 mouse model. The treatment efficacy is compared between intratumoral and subcutaneous administration of SCP. The standard neoantigen vaccine is used with polyIC adjuvant as a positive control. C57BL/6 mice are first seeded with MB49 cells at the flank area. Treatments are given subcutaneously on day 7, and either subcutaneously or intratumorally on days 14 and 17. The exact vaccine contents and administration routes are listed in Table 1 below. Tumour tissues are collected for evaluation on day 27 (FIG. 2A).

TABLE 1

| | Administration content and routes | | |
|---|---|---|---|
| Treatment | Day 7 | Day 14 | Day 17 |
| PBS (sc) | PBS (sc) | PBS (sc) | PBS (sc) |
| PBS (sc + it) | PBS (sc) | PBS (it) | PBS (it) |
| 5 peptides SC | 5 spy-tagged | SCP | SCP |
| phage (sc + it) | MB49 neoantigen | vector | vector |
| | peptides attached to | (without | (it) |
| | the SCP vector | neoantigens) | |
| | (‘5PSCP’) | (it) | |
| | with polyIC (sc) | | |
| 5 peptides + SC | 5 spy-tagged | 5PSCP (it) | 5PSCP (it) |
| phage (sc) | MB49 neoantigen | | |
| | peptides attached to | | |
| | the SCP vector | | |
| | (‘5PSCP’) | | |
| | with polyIC (sc) | | |
| 5 peptides + | 5 MB49 neoantigen | 5P w polyIC | 5P w polyIC |
| polyIC (sc) | peptides (‘5P w | (sc) | (sc) |
| | polyIC’) with polyIC (sc) | | |

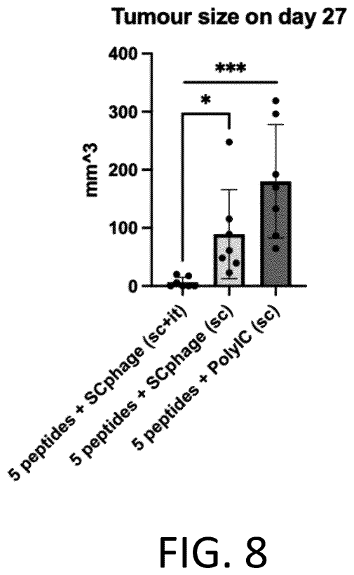

The group treated with subcutaneous neoantigen-linked SCP and intratumoral SCP vaccine showed the best anti-tumour effect, with 4 out of 7 mice experiencing complete tumour regression (FIGS. 2B and 2C). Meanwhile, neoantigen-linked SCP significantly slowed down tumour growth with subcutaneous injection in the 27-day window. Nevertheless, SCP induced a much more significant anti-cancer effect when injected intratumorally, with the mean tumour size on day 27 being 14.4 times smaller (p=0.0147) than when administered purely subcutaneously (FIG. 8). Of note, with the same injection routes, neoantigen-SCP vaccine performed around 2 times better than neoantigen peptide vaccine (p=0.0765). Although the intragroup variation is large, the result suggested that SCP vector could also boost anti-cancer effect by serving as a potent adjuvant like polyIC. Phages may also be able to elicit a more holistic immune response as a viral vector.

Meanwhile, IHC staining demonstrated that intratumoral administration of SCP led to significant increase in CD68+ macrophage infiltration. FIG. 2D showed that the macrophages are densely and diffusely distributed within the tumour tissue, indicating strong immunity being elicited against the tumour cells. To quantify the macrophage infiltration, 5 views are taken on each IHC staining under 20× magnification and normalized the CD68 counts to the total cell number and tissue area. It is found that around 27% of cells within the tumour tissue are CD68 positive in the treated group, which is around 12 times higher than that in the PBS group (p<0.0001) (FIG. 2E). SCP treatment also increased CD68+ infiltration by approximately 10 times compared to PBS-treated group (p<0.0001) when normalized to area. Interestingly, it is also found that CD68 macrophages clustering around large vessels in the tumour margins of the treated mice (FIG. 2F). These results indicated that CD68 cells, as part of the innate immunity, played significant roles in the anti-cancer immunity elicited by intratumoral SCP injection.

On the other hand, CD31 IHC staining revealed that intratumoral phage administration also reduced the size of blood vessels within the tumour mass (FIG. 2G). The morphological changes in intratumoral blood vessels also suggest alternate pathways by which SCP controlled tumour growth, other than inducing robust local immune response.

Meanwhile, SCP treatment also induces significant changes in tumour cells, notably a marked reduction in PDL1 express on MB49 cells (FIG. 4H), while the PDL1 expression slowly recovers after the intratumoral injections are stopped.

Similar phenomenon can also be observed with bacterial lysate injection, but not in mRNA vaccine administration, which could also induce significant local inflammation, suggesting the pathogenic nature of phages is the key in reducing PDL1 expression on tumour cells. Notably, PDL1 expression can be further suppressed by targeting DCs as SCP led to lower expression level compared to spy-catcher phages.

Combining the above results, it is observed that robust systemic tumour-specific immunity and multifaceted modifications to the tumour microenvironment with SCP administration, including inflammation initiation, antigen-specific immune reactions, and restricted blood vessel size. This immune response and cytokine release also reduced PDL1 expression in tumour cells during the early stage of treatment. With this whole-tumour approach, immune response can be tailored to the patient's tumour antigen spectrum. Phages, as immunomodulators, also enhanced immune responses and antigen presentation, potentially leading to more effective cancer treatment outcomes by reprogramming the TME and facilitating tumour resolution.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein are incorporated by reference in their entirety, including all figures and tables, to the extent they are not inconsistent with the explicit teachings of this specification.

It should be understood that numerous specific details, relationships, and methods are set forth to provide a full understanding of the invention. One having ordinary skill in the relevant art, however, will readily recognize that the invention can be practiced without one or more of the specific details or practiced with other methods, protocols, reagents, cell lines and animals. The present invention is not limited by the illustrated ordering of acts or events, as some acts may occur in different orders and/or concurrently with other acts or events. Furthermore, not all illustrated acts, steps or events are required to implement a methodology in accordance with the present invention. Many of the techniques and procedures described, or referenced herein, are well understood and commonly employed using conventional methodology by those skilled in the art.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

1. A dendritic cell-targeting anti-cancer phage immunotherapy composition, comprising a genetically engineered M13 filamentous phage, wherein the genetically engineered phage expresses a dendritic cell-targeting peptide, and a synthetic spy catcher protein.

2. The composition of claim 1, further comprising a neoantigen, wherein the neoantigen is tagged with a synthetic spy tag peptide which forms a fusion protein when presented to the synthetic spy catcher protein.

3. The composition of claim 1, wherein the surface of the genetically engineered phage is further linked with an adjuvant.

4. The composition of claim 3, wherein the adjuvant is selected from tetanus endotoxin, diphtheria toxoid, monophosphoryl lipid A, CpG oligonucleotides, polyinosinic acid-polycytidylic acid (polyIC), saponin-based adjuvants, granulocyte-macrophage colony-stimulating factor (GM-CSF), or combinations thereof.

5. The composition of claim 1, wherein the dendritic cell-targeting peptide comprises an amino acid sequence of SEQ ID NO. 1.

6. The composition of claim 1, wherein the genetically engineering phage expresses the dendritic cell-targeting peptide on its p3 site, and expresses the synthetic spy catcher protein on its p8 site.

7. The composition of claim 2, wherein a linker is provided between the neoantigen and the synthetic spy tag peptide, and the linker is expressed to enhance antigen presentation.

8. The composition of claim 7, wherein the linker comprises an amino acid sequence of SEQ ID NO. 2.

9. A method of treating cancer or cancerous tumour in a subject in need thereof, comprising administering no less than two doses of the composition of claim 1, wherein the administering comprises no less than one subcutaneous administration and one intratumoral administration.

10. The method of claim 9, wherein the tumour size is reduced by 50% within 30 days from the final administration.

11. The method of claim 10, wherein the tumour size reduction effect sustains for at least 60 days since the final administration.

12. The method of claim 9, wherein the subject after total tumour regression shows no recurrence of tumour.


SEQUENCE LISTING (2 sequences)

SEQ ID NO: 1 - protein, 12 aa, synthetic construct
SLSLLTMPGNAS

SEQ ID NO: 2 - protein, 6 aa, synthetic construct
LLSVGG